(19) United States
(12) Patent Application Publication
(10) Pub. No.: US 2024/0221866 A1
(43) Pub. Date: Jul. 4, 2024

(54) METHOD OF REDUCING ARTEFACT VARIANTS IN HIGH THROUGHPUT-SEQUENCING AND USES THEREOF

(71) Applicants: Guangzhou KingMed Transformative Medicine Institute Co., Ltd., Guangzhou, Guangdong (CN); Guangzhou Kingmed Diagnostics Group Co., Ltd., Guangzhou, Guangdong (CN); Guangzhou Kingmed Center for Clinical Laboratory, Guangzhou, Guangdong (CN)

(72) Inventors: Yuhuan MENG, Guangzhou, Guangdong (CN); Guibin LI, Guangzhou, Guangdong (CN); Xiaoqiang HUANG, Guangzhou, Guangdong (CN); Xijie FAN, Guangzhou, Guangdong (CN); Yafei MU, Guangzhou, Guangdong (CN); Xiaping MIAO, Guangzhou, Guangdong (CN); Jiecheng YUAN, Guangzhou, Guangdong (CN); Yating CHENG, Guangzhou, Guangdong (CN); Shihui YU, Guangzhou, Guangdong (CN); Yaoming LIANG, Guangzhou, Guangdong (CN)

(21) Appl. No.: 18/277,111

(22) PCT Filed: Jun. 21, 2021
(86) PCT No.: PCT/CN2021/101184

§ 371 Date: Aug. 14, 2023

(51) Int. Cl. G16B 20/20; G16B 50/30

(52) U.S. Cl. CPC G16B 20/20; G16B 50/30

(57) ABSTRACT

A method of reducing an artefact variant in high-throughput sequencing, which belongs to the technical field of bioinformatics is provided. The method includes the steps of: firstly building a population variant frequency database and integrating to obtain a population variant frequency at each variant site, along with total population, scoring each variant site using the formula designed by the inventors, and dividing a sequence into a predetermined window size after each variant to obtain a plurality of region sequences, then synthetically analyzing the scores of variant sites within each region sequence, and finally excluding the artefact variant sites based on identification. As a result, the method can achieve the detection of all types of artefact variants, and offers advantages such as high efficiency, automation, accuracy and comprehensive detection.

### CROSS-REFERENCE TO RELATED APPLICATIONS

This application claims priority to PCT Application No. PCT/CN2021/101184, having a filing date of Jun. 21, 2021, the entire contents of which are hereby incorporated by reference.

### SEQUENCE LISTING

This application includes a separate sequence listing in compliance with the requirement of 37 C.F.R.§.§ 1.824(a)(2)-1.824 (a)(6) and 1.824 (b), submitted under the file name “0112US01_Sequence listing”, created on Mar. 1, 2024, having a file size of 21 KB, the contents of which are hereby incorporated by reference.

### FIELD OF TECHNOLOGY

The following relates to the technical field of bioinformatics, and particularly, it relates to a method of reducing an artefact variant in high-throughput sequencing and a use thereof.

### BACKGROUND

NGS technology has greatly facilitated gene detection, particularly in whole exome sequencing (WES), which offers cost advantages over complete genome sequencing. WES has gained significant popularity in the medical field due to its high cost-effectiveness.

The primary purpose of WES is to identify pathogenic variants and provide valuable data for disease diagnosis and treatment. However, the variants identified through WES undergo multiple filtration steps for quality control. Despite stringent sequencing quality control measures, some artefacts may still persist due to non-specific amplification and other factors during the PCR amplification process. Such artefacts can have a substantial impact on disease diagnosis.

Presently, there are some methods available to exclude artefacts. However, these methods typically rely on Integrative Genomics viewer (IGV), and necessitate experienced experimenters to make subjective judgment. Therefore, these methods exhibit low efficiency and rely heavily on experimenter expertise, making standardized data processing challenging.

Currently, there is ongoing research on the method of excluding artefact variants, using Loss-Of-Function Transcript Effect Estimator (LOFTEE). This method evaluates stop-gained variants, frameshift variants and alternative splicing variants through a series of filters to identify low-confidence and high-confidence predicted loss-of-function (LOF) variants. However, this method primarily focuses on evaluating and eliminating the aforementioned types of artefacts, potentially overlooking many other sites that may contain artefacts.

### SUMMARY

An aspect relates to providing a method of reducing an artefact variant in high-throughput sequencing. This method aims to automatically identify and exclude artefact variant sites, thereby enhancing sequencing accuracy. The method comprises steps of:
1) building a population variant frequency database comprising: collecting high-throughput sequencing data from a plurality of samples and integrating to obtain a population variant frequency (Fq) at each variant site, along with total population (Pop);
2) scoring a variant site comprising: obtaining high-throughput sequencing data from samples to be tested, then merging information from each variant site, and scoring each variant site using the following formula:

$$Svar = {( Fq \times Pop \times 2 )}_{max = M} \times ( Score 1 + Score 2 + Score 3 )$$

wherein Svar represents a score of the variant site, Fq represents a population variant frequency, Pop represents a total population, M represents a pre-determined maximum value, Score1 represents a variant type score, Score2 represents a variant coordinate score, Score3 represents a pLoF score;
Score1 is evaluated based on the following standard: if a variant is known, Score1 is assigned a value of 0. However, if the variant is unknown, Score1 is assigned a negative value;
Score2 is evaluated based on the following standard: if the variant is unknown, Score2 is assigned a negative value according to a pre-set rule based on a location of the variant within a refGene region;
Score3 is evaluated based on the following standard: Score3 is assigned a positive value based on a predefined rule that determines a confidence level of predicted loss-of-function (LOF) variants filtered using LOFTEE;
3) scoring region sequence comprising: dividing a sequence into a predetermined window size after each variant to obtain a plurality of region sequences comprising variant sites,
wherein each region sequence is evaluated using the following formula:

$$S total = N \times {\sum }_{k = 0}^{n} Svar$$

wherein Stotal represents a total score of the region sequence, N represents a total number of variants within the region sequence; and
4) excluding a variant site: if the total score of the region sequence (Stotal) is lower than a predetermined threshold value, a variant site within the region sequence is determined to be an artefact variant site and is excluded.

Based on extensive practical work, the inventors have carefully analyzed, deduced and confirmed that the artefact variant comprises the features as follows: Specifically, a significant number of insertion-deletion variants (INDELs) tend to cluster at both ends of captured sequencing regions. Different experimenters make varying judgements on insertion-deletion variants (INDELs) within a particular region, resulting in a high occurrence of INDELs in the population. Building upon previous research, the inventors of the present disclosure propose a method of massively excluding artefact variants. This method comprises the steps of scoring variant sites within a specific window length, weighting scores of the variant sites, obtaining a region sequence score within the window, and screening the variant sites within a confidence interval to massively exclude the artefact variants.

The above method comprises the steps of: firstly building a population variant frequency database and integrating to obtain a population variant frequency at each variant site, along with total population (Pop), scoring each variant site using the formula designed by the inventors, and dividing a sequence into a predetermined window size after each variant to obtain a plurality of region sequences, then synthetically analyzing the scores of variant sites within each region sequence, and finally excluding the artefact variant sites based on identification. In the step of synthetically analyzing the scores of variant sites within each region sequence, considering the occurrence of variants within a specific window size after each variant site, the scores of all variants within the region sequence are summed up, and the sum is multiplied by the number of variants in the window for amplification, thereby highlighting the artefact variant sites. As a result, the method can achieve the detection of all types of artefact variants, and offers advantages such as high efficiency, automation, accuracy and comprehensive detection.

In one example, in the step of building a population variant frequency database, each variant site is sorted based on chromosome and genome position.

In one example, in the step of scoring a variant site, Score1 is evaluated based on the following standard: if a variant site has an annotation in a database selected from a group consisting of dbSNP, ClinVar, and geomAD-exome, the variant site is determined to be known and Score1 is assigned a value of zero; and if the variant site lacks such annotation, the variant site is determined to be unknown, and Score1 is assigned a negative value;
Score2 is evaluated based on the following standard: if the variant is unknown and falls within a region selected from a group consisting of exonic region, splicing region, UTR-3 region, UTR-5 region, upstream region, downstream region, intronic region, intergenic region, and ncRNA region in refGene, Score2 is assigned a negative value according to the pre-set rule; and
Score3 is evaluated based on the following standard: if the variant is determined to be a high-confidence or low-confidence predicted loss-of-function (pLoF) variant filtered using LOFTEE, Score3 is assigned a weighted value determined by a predetermined rule.

It should be noted that, the above-mentioned “dbSNP” refers to “the single nucleotide polymorphism database”, representing “a single base-pair variation (A, T, C, G) in DNA sequence”. It signifies the presence of two or more potential nucleotides are in genome, which is the most common naturally occurring variation in humans. Known variants in the technical field are categorized based on their location and variant type, aiding in determining whether the variant is an artefact. In one example, the dbSNP database is dbSNP150. Additionally, the above-mentioned predicted loss-of-function (pLoF) variant filtered using LOFTEE refers to a processed predicted loss of function (pLoF) variant in the haploid disease gene within the Genome Aggregation Database (gnomAD), aiding in distinguishing this variant type for subsequent analysis and reference.

In one example, in the step of scoring a variant site:
Score1 is evaluated based on the following standard: if the variant is known, Score1 is assigned a value of 0; if the variant is an unknown single nucleotide variant (SNV), Score1 is assigned a value of −1; if the variant is an unknown insertion-deletion variant (INDEL), Score1 is assigned a value of −5;
Score2 is evaluated based on the following standard: if the variant is located in an exonic region, Score2 is assigned a value of 0; if the variant is located in a region selected from a group consisting of UTR-3 region, UTR-5 region, upstream region, downstream region, intronic region, intergenic region, and ncRNA region, Score2 is assigned a value of −1; if the variant is located in a splicing region, Score2 is assigned a value of −2; and
Score3 is evaluated based on the following standard: if the variant is a high-confidence predicted loss-of-function (pLoF) variant, Score3 is assigned a value of +3; if the variant is a low-confidence pLoF variant, Score3 is assigned a value of +2.

The weighting scores above are designed to address the main features of the artefacts, particularly the clustering of false positive INDELs at both ends of the captured sequencing region). In this context, the highest weighting is given to INDELs within a specific window, and thereby detecting the highest weighting of unknown INDELs within the window. Other factors taken into account include SNP, specially, whether they are known or unknown, the type of variant, and whether a pLoF variant is annotated in LOEFF, etc. Through tests, it has been determined that the defined weighting for each factor effectively identifies artefact variants well.

In one example, in the step of scoring a variant site, a value of M is 100.

To clarify, a known variant refers to a reported variant. For the population variant frequency, obtaining specific population data for a variant site in practice can be challenged. Therefore, a frequency measure is generally used to gauge the occurrence of the variant site. When the frequency multiplied by the population and 2 exceeds 100, it indicates that the variant site is well-known. As a result, a maximum value of 100, denoted as M is established.

In one example, in the step of scoring a variant site, if the score of the variant site (Svar) is greater than 0, Svar is assigned a value of 0. Considering the scoring system used to evaluate variant sites in this method, if the score of the variant site (Svar) is greater than 0, that is, Svar is positive (not negative), Svar is then assigned a value of 0. This adjustment allows for a more accurate evaluation of whether an artefact variant is present in a region sequence.

In one example, the position of refGene is determined by aligning it with the master transcript region of NCBI refGene. For pLoF variants, only Stop-gained variant sites, Splicing variant sites, and Frameshift variant sites found in the master transcript are retained.

In one example, the master transcript refers to the most recently updated transcript in the NCBI refGene, specifically the transcript with the shortest time span from its creation to the current time.

In one example, in the step of scoring the region sequence, the window size is determined as follows: the window size corresponds to a length of a fragment interval that covers 95±5% of adjacent variant sites in the database. The “adjacent” herein refers to fragment intervals with consecutive lengths for the two adjacent variant sites. By setting the window size to cover the majority of the fragment interval for two adjacent variant sites, artefact variants can be accurately identified.

In one example, the database is dbSNP database, ECAC03 whole exon database and/or genomeAD whole exon database.

In one example, the window size is set to 25±5 base pairs (bp).

In the previous study, the inventors analyzed and compared the dbSNP150, EXAC03 whole exon database, and genomeAD whole exon database. They discovered that a window size of 25 bp could cover over 95% of fragment intervals.

In one example, in the step of excluding a variant site, the predetermined threshold value is determined as the top 5% total score (Stotal) of the region sequence when sorting the total score (Stotal) of each region sequence in a sample to be tested from low to high. It should be understood that, the threshold value can also be adjusted to a specific score threshold based on a practical requirements.

Another aspect relates to providing use of the method of reducing an artefact variant in high-throughput sequencing.

In one example, the high-throughput sequencing is whole exome sequencing.

By applying the above-mentioned method of reducing an artefact variant in high-throughput sequencing, particularly in whole exome sequencing, artefact variants can be minimized, providing accurate data for disease diagnosis and improving the reliability of personalized precision medicine.

Another aspect relates to providing use of the above-mentioned method in a diagnosis and treatment of disease, as well as variant screening of whole exome.

Another aspect relates to providing a device for reducing an artefact variant in high-throughput sequencing, comprising:
a module of analysis, configured for analyzing variant data obtained from the high-throughput sequencing data of a sample to be tested using the above-mentioned method of reducing an artefact variant in high-throughput sequencing.

It should be noted that the above-mentioned device can in a form of a product, such as an integrated device, or a software module wrapper, used for analysis based on the above method.

Compared to the conventional art, embodiments of the present invention have the advantages as follows:

In embodiments, the method of reducing an artefact variant in high-throughput sequencing of the present disclosure can identify and exclude low frequency artefact variants, harmful artefact variants, as well as general artefact variants, making it comprehensive and accurate.

In embodiments, the method of the present disclosure allows for automatic data analysis and processing using computers, overcoming the defeats of artificial exclusion, such as, low efficiency and dependency on subjective experience. As a result, it is suitable for widespread adoption and application.

### BRIEF DESCRIPTION

Some of examples will be described in detail, with reference to the following figures, wherein like designations denote like members, wherein:

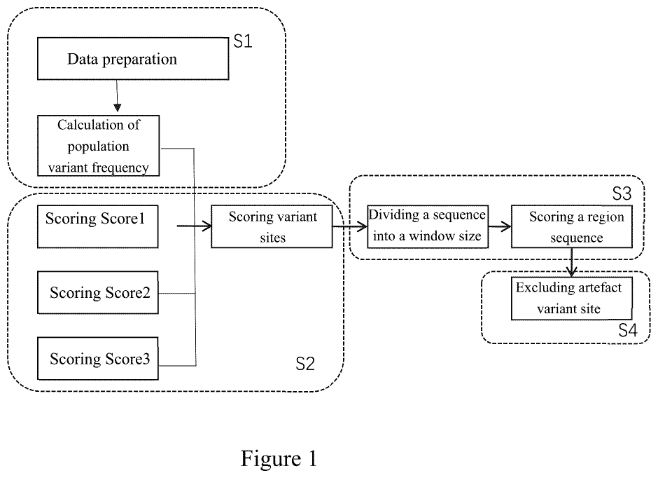

FIG. 1 depicts a flow chart of the method of reducing an artefact variant in high-throughput sequencing, as demonstrated in Examples;

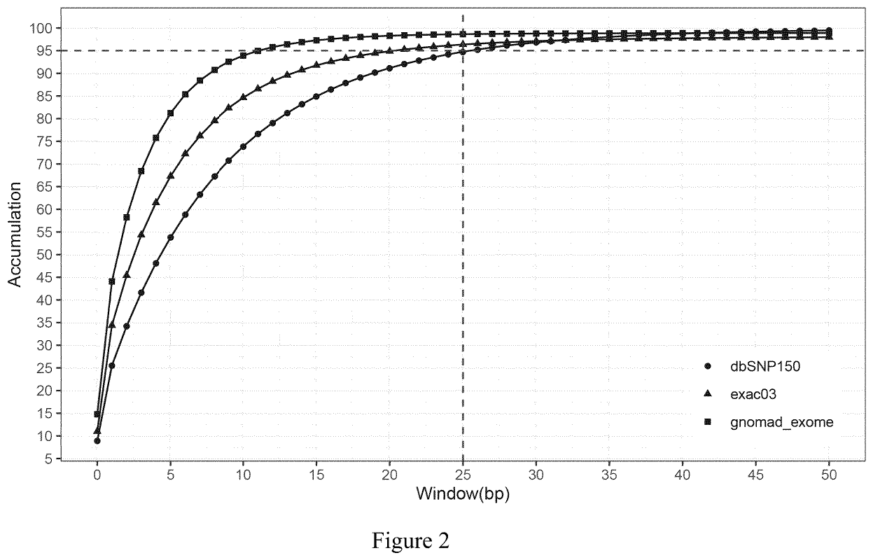

FIG. 2 depicts a schematic diagram of the Variants internal concerning different window sizes, as observed in the Examples;

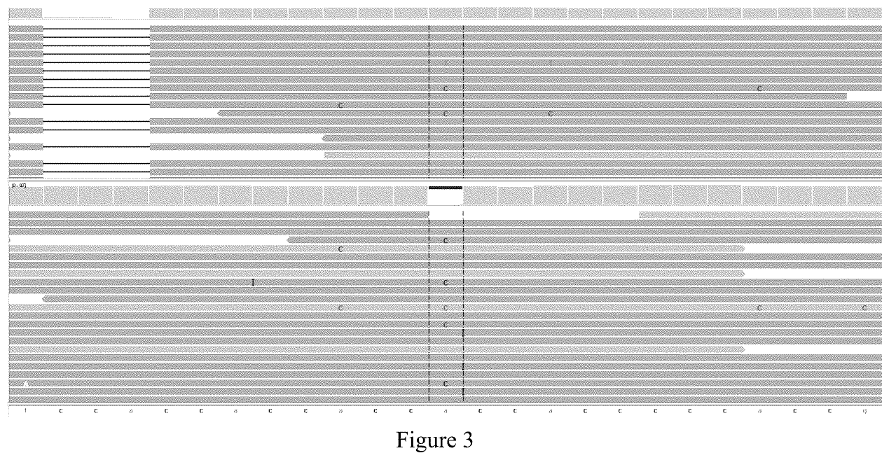

FIG. 3 depicts a IGV visualization result for the site chr9: 35906583 in the Examples; and

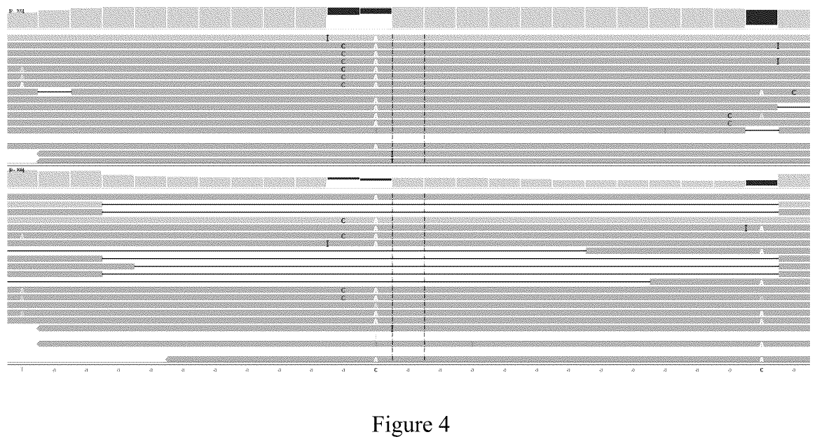

FIG. 4 depicts a IGV visualization result for the site chr9: 32986030 in the Examples.

### DETAILED DESCRIPTION

To facilitate a comprehensive understanding of the present disclosure, relevant drawings will be utilized to describe the present disclosure. These drawings depict certain embodiments of the present disclosure. However, it should be noted that the present disclosure can be implemented in various forms and is not limited to the embodiments described herein. The embodiments are provided to ensure a thorough and complete contents of the present disclosure.

Unless otherwise defined, all technical and scientific terms used herein hold the same meaning as those normally understood by one skilled in the conventional art in the technical field to which the present disclosure belongs. The terms used throughout the description of the present disclosure herein are solely intended to describe specific embodiments, and should not be construed as limiting the scope of the present disclosure. The term “and/or” used herein refers to any one or more relevant items and their combination.

### Example

A method of reducing an artefact variant in high-throughput sequencing comprised the following steps, with reference to FIG. 1:

### 1. Building a Population Variant Frequency Database

High-throughput sequencing data were collected from a plurality of samples and integrated to determine a population variant frequency at each variant site. The process comprised the following steps:

### 1.1 Data Preparation

High-throughput sequencing data were obtained from a plurality of samples, (e.g., 42868 samples in the Example). A VCF (Variant Call Format) merge file comprising the information from all the sites was obtained. The information from each variant site comprised a chromosome, a specific position, refGenome bases, variant base types, and other relevant details. These details were sorted based on the chromosome and genome position.

### 1.2 Calculation of Population Variant Frequency

The population variant frequency, a crucial parameter for calculating the evaluating scores of the variant sites in the present method of the present disclosure, was determined through calculation. The information from the variant sites mentioned above were formatted according to the annovar standard, wherein frequency data was added to the VCF format file. A partial representation of this data was provided in the table below.

TABLE 1
Input File Format for Population Variant and Frequency

| | | Reference | Variant | Allele | | Homozygous/ |
|---|---|---|---|---|---|---|
| Chromosome | Position | base | base | frequency | Information | Heterozygous |
| chr1 | 14653 | C | T | 2.33263e−05 | GT | 1/1 |
| chr1 | 14907 | A | G | 0.000128295 | GT | 1/1 |
| chr1 | 14930 | A | G | 0.000139958 | GT | 1/1 |
| chr1 | 14932 | G | T | 2.33263e−05 | GT | 1/1 |
| chr1 | 14933 | G | A | 1.16632e−05 | GT | 1/1 |
| chr1 | 15903 | G | GC | 6.9979e−05 | GT | 1/1 |
| chr1 | 69331 | C | T | 1.16632e−05 | GT | 1/1 |
| chr1 | 69336 | C | T | 1.16632e−05 | GT | 1/1 |
| chr1 | 69460 | C | T | 1.16632e−05 | GT | 1/1 |
| chr1 | 69462 | C | G | 2.33263e−05 | GT | 1/1 |
| chr1 | 69474 | T | G | 1.16632e−05 | GT | 1/1 |
| chr1 | 69486 | C | A | 2.33263e−05 | GT | 1/1 |
| chr1 | 69486 | C | T | 2.33263e−05 | GT | 1/1 |
| chr1 | 69491 | G | A | 1.16632e−05 | GT | 1/1 |
| chr1 | 69495 | C | T | 1.16632e−05 | GT | 1/1 |
| chr1 | 69496 | G | A | 1.16632e−05 | GT | 1/1 |
| chr1 | 69496 | G | T | 8.16422e−05 | GT | 1/1 |
| chr1 | 69510 | C | T | 1.16632e−05 | GT | 1/1 |
| chr1 | 69511 | A | G | 0.942186 | GT | 1/1 |
| chr1 | 69513 | A | G | 0.000606485 | GT | 1/1 |
| chr1 | 69521 | T | C | 8.16422e−05 | GT | 1/1 |
| chr1 | 69522 | T | C | 1.16632e−05 | GT | 1/1 |
| chr1 | 69534 | T | C | 0.00271752 | GT | 1/1 |
| chr1 | 69543 | C | A | 1.16632e−05 | GT | 1/1 |
| chr1 | 69550 | G | A | 9.33053e−05 | GT | 1/1 |
| chr1 | 69550 | G | T | 1.16632e−05 | GT | 1/1 |
| chr1 | 69552 | G | A | 1.16632e−05 | GT | 1/1 |
| chr1 | 69555 | T | G | 1.16632e−05 | GT | 1/1 |
| chr1 | 69559 | G | A | 0.000314906 | GT | 1/1 |
| chr1 | 69563 | A | C | 6.9979e−05 | GT | 1/1 |

The above table provided a list of the input file format for population variant and frequency. It included the following columns:
Chromosome and position represented the position of the variant in genome,
Reference Base referred to a base type of the reference genome hg19,
Variant Base represented a base type detected in this sequencing that differed from reference genome,
Allele Frequency represented the variant frequency in a population, calculated as the number of variants divided by the total population multiplied by 2;
GT: short for Genetype in the list of information, represented genotype of variant;
1/1 represented homozygous genotype,
0/1 represented heterozygous genotype.

The VCF format was converted to the annovar standard format using the built in function module “convert2annovar.pl-format vcf4” in the annovar software. Examples of the converted format were provided in Table 2.

TABLE 2
Input file format of data on variant and frequency before annotated

| | Initiation | Termination | Reference | Variant | Homozygous/ | |
|---|---|---|---|---|---|---|
| Chromosome | position | position | base | base | Heterozygous | Frequency |
| chr1 | 14653 | 14653 | C | T | het | 2.33263e−05 |
| chr1 | 14907 | 14907 | A | G | het | 0.000128295 |
| chr1 | 14930 | 14930 | A | G | het | 0.000139958 |
| chr1 | 14932 | 14932 | G | T | het | 2.33263e−05 |
| chr1 | 14933 | 14933 | G | A | het | 1.16632e−05 |
| chr1 | 15903 | 15903 | — | C | het | 6.9979e−05 |
| chr1 | 69331 | 69331 | C | T | het | 1.16632e−05 |
| chr1 | 69336 | 69336 | C | T | het | 1.16632e−05 |
| chr1 | 69460 | 69460 | C | T | het | 1.16632e−05 |
| chr1 | 69462 | 69462 | C | G | het | 2.33263e−05 |
| chr1 | 69474 | 69474 | T | G | het | 1.16632e−05 |
| chr1 | 69486 | 69486 | C | A | het | 2.33263e−05 |
| chr1 | 69486 | 69486 | C | T | het | 2.33263e−05 |
| chr1 | 69491 | 69491 | G | A | het | 1.16632e−05 |
| chr1 | 69495 | 69495 | C | T | het | 1.16632e−05 |
| chr1 | 69496 | 69496 | G | A | het | 1.16632e−05 |
| chr1 | 69496 | 69496 | G | T | het | 8.16422e−05 |
| chr1 | 69510 | 69510 | C | T | het | 1.16632e−05 |
| chr1 | 69511 | 69511 | A | G | het | 0.942186 |
| chr1 | 69513 | 69513 | A | G | het | 0.000606485 |
| chr1 | 69521 | 69521 | T | C | het | 8.16422e−05 |
| chr1 | 69522 | 69522 | T | C | het | 1.16632e−05 |
| chr1 | 69534 | 69534 | T | C | het | 0.00271752 |
| chr1 | 69543 | 69543 | C | A | het | 1.16632e−05 |
| chr1 | 69550 | 69550 | G | A | het | 9.33053e−05 |
| chr1 | 69550 | 69550 | G | T | het | 1.16632e−05 |
| chr1 | 69552 | 69552 | G | A | het | 1.16632e−05 |
| chr1 | 69555 | 69555 | T | G | het | 1.16632e−05 |
| chr1 | 69559 | 69559 | G | A | het | 0.000314906 |
| chr1 | 69563 | 69563 | A | C | het | 6.9979e−05 |

### 2. Scoring the Variant Sites (S2)

The high-throughput sequencing data of samples to be tested were obtained to gain information of each variant site. Each variant site was scored using the following formula:

Svar=(Fq×Pop×2)ₘₐₓ₌M×(Score1+Score2+Score3)

wherein, Svar represented a score of the variant site, Fq represented a population variant frequency, Pop represented a total population, M represented a pre-determined maximum value, Score1 represented a variant type score, Score2 represented a variant coordinate score, Score3 represented a pLoF score.

Specifically, S2 comprised the following sub-steps:

### 2.1 Scoring Score1

The population variant data comprising the population variant frequency was annotated using annovoar. The annotation was based on database such as dbSNP150, ClinVar database and gnomAD-exome database.

If the variant site was annotated in any one of dbSNP150, ClinVar or gnomAD-exome database, the variant of the variant site was determined to be a known variant, resulting in a Score1 of 0; if the variant was determined to be an unknown single nucleotide variant (SNV), resulting in a Score1 of −1; if the variant was determined to be an unknown insertion-deletion variant (INDEL), resulting in a Score1 of −5.

### 2.2 Scoring Score2

Based on the Score1 result, Score2 was not evaluated for known variants; Score2 was evaluated for unknown variants according to their location in the refGene. For example, if the variant was located in exonic region, Score2 was assigned a value of 0; if the variant was found in a region selected from a group consisting of UTR-3 region, TUR-5 region, upstream region, downstream region, intronic region, intergenic region, and ncRNA region, Score2 was assigned a value of −1; if the variant was located in a splicing region, Score2 was assigned a value of −2.

In this Example, the annotation using annovar in the Steps of scoring Score1 and scoring Score2 above were performed on Ubuntu 18.04.2 LTS, using the following command: able_annovar.pl all.vcf.avinput dir/humandb/-buildver hg19-out x-otherinfo-remove-protocol refGene,avsnp150,clinvar_20200316,gnomad_exome-operation g,f,f,f-nastring NA.

### 2.3 Scoring Score3

Predicted loss-of-function (pLoF) variants in haploid disease genes were identified, and the resulting pLoF variant data were screened. The pLoF sites were annotated using annovar, based on the master transcript of the gene, which represented the most recently updated transcript of the gene in the NCBI refGene. The annotation included information, such as the position of the pLoF site in the genome, the specific transcript within the gene, variation types, and possible amino acid variants.

Some examples of Annovar annotations were shown as follows.

TABLE 3
Annotation results from annovar (refGene annotation)

| | | | | | Annotation | | Functional | | |
|---|---|---|---|---|---|---|---|---|---|
| | | | | | result | | variant | Variant | |
| | Initiation | Termination | Reference | Variant | of | Corresponding | types | in amino | Allele |
| Chromosome | position | position | base | base | refGene | gene | of exon | acids | frequency |
| chr1 | 14653 | 14653 | C | T | ncRNA_exonic | WASH7P | NA | NA | 2.33263e−05 |
| chr1 | 14907 | 14907 | A | G | ncRNA_intronic | WASH7P | NA | NA | 0.000128295 |
| chr1 | 14930 | 14930 | A | G | ncRNA_intronic | WASH7P | NA | NA | 0.000139958 |
| chr1 | 14932 | 14932 | G | T | ncRNA_intronic | WASH7P | NA | NA | 2.33263e−05 |
| chr1 | 14933 | 14933 | G | A | ncRNA_intronic | WASH7P | NA | NA | 1.16632e−05 |
| chr1 | 15903 | 15903 | — | C | ncRNA_exonic | WASH7P | NA | NA | 6.9979e−05 |
| chr1 | 69331 | 69331 | C | T | exonic | OR4F5 | stopgain | OR4F5: | 1.16632e−05 |
| | | | | | | | | NM_001005484: | |
| | | | | | | | | exon1: | |
| | | | | | | | | c.C241T: | |
| | | | | | | | | p.Q81X | |
| chr1 | 69336 | 69336 | C | T | exonic | OR4F5 | synonymous | OR4F5: | 1.16632e−05 |
| | | | | | | | SNV | NM_001005484: | |
| | | | | | | | | exon1: | |
| | | | | | | | | c.C246T: | |
| | | | | | | | | p.R82R | |
| chr1 | 69460 | 69460 | C | T | exonic | OR4F5 | nonsynonymous | OR4F5: | 1.16632e−05 |
| | | | | | | | SNV | NM_001005484: | |
| | | | | | | | | exon1: | |
| | | | | | | | | c.C370T: | |
| | | | | | | | | p.H124Y | |
| chr1 | 69462 | 69462 | C | G | exonic | OR4F5 | nonsynonymous | OR4F5: | 2.33263e−05 |
| | | | | | | | SNV | NM_001005484: | |
| | | | | | | | | exon1: | |
| | | | | | | | | c.C372G: | |
| | | | | | | | | p.H124Q | |
| chr1 | 69474 | 69474 | T | G | exonic | OR4F5 | nonsynonymous | OR4F5: | 1.16632e−05 |
| | | | | | | | SNV | NM_001005484: | |
| | | | | | | | | exon1: | |
| | | | | | | | | c.T384G: | |
| | | | | | | | | p.I128M | |
| chr1 | 69486 | 69486 | C | A | exonic | OR4F5 | nonsynonymous | OR4F5: | 2.33263e−05 |
| | | | | | | | SNV | NM_001005484: | |
| | | | | | | | | exon1: | |
| | | | | | | | | c.C396A: | |
| | | | | | | | | p.N132K | |
| chr1 | 69486 | 69486 | C | T | exonic | OR4F5 | synonymous | OR4F5: | 2.33263e−05 |
| | | | | | | | SNV | NM_001005484: | |
| | | | | | | | | exon1: | |
| | | | | | | | | c.C396T: | |
| | | | | | | | | p.N132N | |
| chr1 | 69491 | 69491 | G | A | exonic | OR4F5 | nonsynonymous | OR4F5: | 1.16632e−05 |
| | | | | | | | SNV | NM_001005484: | |
| | | | | | | | | exon1: | |
| | | | | | | | | c.G401A: | |
| | | | | | | | | p.C134Y | |
| chr1 | 69495 | 69495 | C | T | exonic | OR4F5 | synonymous | OR4F5: | 1.16632e−05 |
| | | | | | | | SNV | NM_001005484: | |
| | | | | | | | | exon1: | |
| | | | | | | | | c.C405T: | |
| | | | | | | | | p.V135V | |
| chr1 | 69496 | 69496 | G | A | exonic | OR4F5 | nonsynonymous | OR4F5: | 1.16632e−05 |
| | | | | | | | SNV | NM_001005484: | |
| | | | | | | | | exon1: | |
| | | | | | | | | c.G406A: | |
| | | | | | | | | p.G136S | |
| chr1 | 69496 | 69496 | G | T | exonic | OR4F5 | nonsynonymous | OR4F5: | 8.16422e−05 |
| | | | | | | | SNV | NM_001005484: | |
| | | | | | | | | exon1: | |
| | | | | | | | | c.G406T: | |
| | | | | | | | | p.G136C | |
| chr1 | 69510 | 69510 | C | T | exonic | OR4F5 | synonymous | OR4F5: | 1.16632e−05 |
| | | | | | | | SNV | NM_001005484: | |
| | | | | | | | | exon1: | |
| | | | | | | | | c.C420T: | |
| | | | | | | | | p.V140V | |
| chr1 | 69511 | 69511 | A | G | exonic | OR4F5 | nonsynonymous | OR4F5: | 0.942186 |
| | | | | | | | SNV | NM_001005484: | |
| | | | | | | | | exon1: | |
| | | | | | | | | c.A421G: | |
| | | | | | | | | p.T141A | |
| chr1 | 69513 | 69513 | A | G | exonic | OR4F5 | synonymous | OR4F5: | 0.000606485 |
| | | | | | | | SNV | NM_001005484: | |
| | | | | | | | | exon1: | |
| | | | | | | | | c.A423G: | |
| | | | | | | | | p.T141T | |
| chr1 | 69521 | 69521 | T | C | exonic | OR4F5 | nonsynonymous | OR4F5: | 8.16422e−05 |
| | | | | | | | SNV | NM_001005484: | |
| | | | | | | | | exon1: | |
| | | | | | | | | c.T431C: | |
| | | | | | | | | p.I144T | |
| chr1 | 69522 | 69522 | T | C | exonic | OR4F5 | synonymous | OR4F5: | 1.16632e−05 |
| | | | | | | | SNV | NM_001005484: | |
| | | | | | | | | exon1: | |
| | | | | | | | | c.T432C: | |
| | | | | | | | | p.I144I | |
| chr1 | 69534 | 69534 | T | C | exonic | OR4F5 | synonymous | OR4F5: | 0.00271752 |
| | | | | | | | SNV | NM_001005484: | |
| | | | | | | | | exon1: | |
| | | | | | | | | c.T444C: | |
| | | | | | | | | p.H148H | |
| chr1 | 69543 | 69543 | C | A | exonic | OR4F5 | nonsynonymous | OR4F5: | 1.16632e−05 |
| | | | | | | | SNV | NM_001005484: | |
| | | | | | | | | exon1: | |
| | | | | | | | | c.C453A: | |
| | | | | | | | | p.S151R | |
| chr1 | 69550 | 69550 | G | A | exonic | OR4F5 | nonsynonymous | OR4F5: | 9.33053e−05 |
| | | | | | | | SNV | NM_001005484: | |
| | | | | | | | | exon1: | |
| | | | | | | | | c.G460A: | |
| | | | | | | | | p.A154T | |
| chr1 | 69550 | 69550 | G | T | exonic | OR4F5 | nonsynonymous | OR4F5: | 1.16632e−05 |
| | | | | | | | SNV | NM001005484: | |
| | | | | | | | | exon1: | |
| | | | | | | | | c.G460T: | |
| | | | | | | | | p.A154S | |
| chr1 | 69552 | 69552 | G | A | exonic | OR4F5 | synonymous | OR4F5: | 1.16632e−05 |
| | | | | | | | SNV | NM_001005484: | |
| | | | | | | | | exon1: | |
| | | | | | | | | c.G462A: | |
| | | | | | | | | p.A154A | |
| chr1 | 69555 | 69555 | T | G | exonic | OR4F5 | nonsynonymous | OR4F5: | 1.16632e−05 |
| | | | | | | | SNV | NM_001005484: | |
| | | | | | | | | exon1: | |
| | | | | | | | | c.T465G: | |
| | | | | | | | | p.F155L | |
| chr1 | 69559 | 69559 | G | A | exonic | OR4F5 | nonsynonymous | OR4F5: | 0.000314906 |
| | | | | | | | SNV | NM_001005484: | |
| | | | | | | | | exon1: | |
| | | | | | | | | c.G469A: | |
| | | | | | | | | p.V157M | |

TABLE 4
Annotation results from annovar (annotation
results from dbSNP 150)

| | | | | | Annotation |
|---|---|---|---|---|---|
| | Initiation | Terminate | Reference | Variant | result from |
| Chromosome | position | position | base | base | dbSNP150 |
| Chr | Start | End | Ref | Alt | avsnp150 |
| chr1 | 14653 | 14653 | C | T | rs62635297 |
| chr1 | 14907 | 14907 | A | G | rs6682375 |
| chr1 | 14930 | 14930 | A | G | rs6682385 |
| chr1 | 14932 | 14932 | G | T | NA |
| chr1 | 14933 | 14933 | G | A | rs199856693 |
| chr1 | 15903 | 15903 | — | C | rs557514207 |
| chr1 | 69331 | 69331 | C | T | NA |
| chr1 | 69336 | 69336 | C | T | NA |
| chr1 | 69460 | 69460 | C | T | NA |
| chr1 | 69462 | 69462 | C | G | NA |
| chr1 | 69474 | 69474 | T | G | rs752034042 |
| chr1 | 69486 | 69486 | C | A | NA |
| chr1 | 69486 | 69486 | C | T | rs548369610 |
| chr1 | 69491 | 69491 | G | A | NA |
| chr1 | 69495 | 69495 | C | T | NA |
| chr1 | 69496 | 69496 | G | A | rs150690004 |
| chr1 | 69496 | 69496 | G | T | NA |
| chr1 | 69510 | 69510 | C | T | NA |
| chr1 | 69511 | 69511 | A | G | rs2691305 |
| chr1 | 69513 | 69513 | A | G | rs770590115 |
| chr1 | 69521 | 69521 | T | C | rs553724620 |
| chr1 | 69522 | 69522 | T | C | NA |
| chr1 | 69534 | 69534 | T | C | rs190717287 |
| chr1 | 69543 | 69543 | C | A | NA |
| chr1 | 69550 | 69550 | G | A | NA |
| chr1 | 69550 | 69550 | G | T | NA |
| chr1 | 69552 | 69552 | G | A | rs2531266 |
| chr1 | 69555 | 69555 | T | G | rs556374459 |
| chr1 | 69559 | 69559 | G | A | rs754025211 |

TABLE 5
Annotation results from annovar (annotation results from ClinVar)

| | Initiation | Terminate | Reference | Variant | annotation details from ClinVar | | | | |
|---|---|---|---|---|---|---|---|---|---|
| Chromosome | position | position | base | base | CLNALLELEID | CLNDN | CLNDISDB | CLNREVSTAT | CLNSIG |
| chr1 | 14653 | 14653 | C | T | NA | NA | NA | NA | NA |
| chr1 | 14907 | 14907 | A | G | NA | NA | NA | NA | NA |
| chr1 | 14930 | 14930 | A | G | NA | NA | NA | NA | NA |
| chr1 | 14932 | 14932 | G | T | NA | NA | NA | NA | NA |
| chr1 | 14933 | 14933 | G | A | NA | NA | NA | NA | NA |
| chr1 | 15903 | 15903 | — | C | NA | NA | NA | NA | NA |
| chr1 | 69331 | 69331 | C | T | NA | NA | NA | NA | NA |
| chr1 | 69336 | 69336 | C | T | NA | NA | NA | NA | NA |
| chr1 | 69460 | 69460 | C | T | NA | NA | NA | NA | NA |
| chr1 | 69462 | 69462 | C | G | NA | NA | NA | NA | NA |
| chr1 | 69474 | 69474 | T | G | NA | NA | NA | NA | NA |
| chr1 | 69486 | 69486 | C | A | NA | NA | NA | NA | NA |
| chr1 | 69486 | 69486 | C | T | NA | NA | NA | NA | NA |
| chr1 | 69491 | 69491 | G | A | NA | NA | NA | NA | NA |
| chr1 | 69495 | 69495 | C | T | NA | NA | NA | NA | NA |
| chr1 | 69496 | 69496 | G | A | NA | NA | NA | NA | NA |
| chr1 | 69496 | 69496 | G | T | NA | NA | NA | NA | NA |
| chr1 | 69510 | 69510 | C | T | NA | NA | NA | NA | NA |
| chr1 | 69511 | 69511 | A | G | NA | NA | NA | NA | NA |
| chr1 | 69513 | 69513 | A | G | NA | NA | NA | NA | NA |
| chr1 | 69521 | 69521 | T | C | NA | NA | NA | NA | NA |
| chr1 | 69522 | 69522 | T | C | NA | NA | NA | NA | NA |
| chr1 | 69534 | 69534 | T | C | NA | NA | NA | NA | NA |
| chr1 | 69543 | 69543 | C | A | NA | NA | NA | NA | NA |
| chr1 | 69550 | 69550 | G | A | NA | NA | NA | NA | NA |
| chr1 | 69550 | 69550 | G | T | NA | NA | NA | NA | NA |
| chr1 | 69552 | 69552 | G | A | NA | NA | NA | NA | NA |
| chr1 | 69555 | 69555 | T | G | NA | NA | NA | NA | NA |
| chr1 | 69559 | 69559 | G | A | NA | NA | NA | NA | NA |

TABLE 6
annovar annotation results (exome annotation results from gnomAD)

| | Initia- | Termi- | | | | | | | | | | | |
|---|---|---|---|---|---|---|---|---|---|---|---|---|---|
| Chromo- | tion | nation | Reference | Variant | Exome annotation details from gnomAD_{—} | | | | | | | | |
| some | site | site | base | base | ALL | AFR | AMR | ASJ | EAS | FIN | NFE | OTH | SAS |
| chr1 | 14653 | 14653 | C | T | NA | NA | NA | NA | NA | NA | NA | NA | NA |
| chr1 | 14907 | 14907 | A | G | NA | NA | NA | NA | NA | NA | NA | NA | NA |
| chr1 | 14930 | 14930 | A | G | NA | NA | NA | NA | NA | NA | NA | NA | NA |
| chr1 | 14932 | 14932 | G | T | NA | NA | NA | NA | NA | NA | NA | NA | NA |
| chr1 | 14933 | 14933 | G | A | NA | NA | NA | NA | NA | NA | NA | NA | NA |
| chr1 | 15903 | 15903 | — | C | NA | NA | NA | NA | NA | NA | NA | NA | NA |
| chr1 | 69331 | 69331 | C | T | NA | NA | NA | NA | NA | NA | NA | NA | NA |
| chr1 | 69336 | 69336 | C | T | NA | NA | NA | NA | NA | NA | NA | NA | NA |
| chr1 | 69460 | 69460 | C | T | NA | NA | NA | NA | NA | NA | NA | NA | NA |
| chr1 | 69462 | 69462 | C | G | NA | NA | NA | NA | NA | NA | NA | NA | NA |
| chr1 | 69474 | 69474 | T | G | 1.234e−05 | 0.0002 | 0 | 0 | 0 | 0 | 0 | 0 | 0 |
| chr1 | 69486 | 69486 | C | A | 6.193e−06 | 0 | 0 | 0 | 5.984e−05 | 0 | 0 | 0 | 0 |
| chr1 | 69486 | 69486 | C | T | 2.477e−05 | 0 | 0 | 0 | 0 | 0 | 5.737e−05 | 0 | 0 |
| chr1 | 69491 | 69491 | G | A | NA | NA | NA | NA | NA | NA | NA | NA | NA |
| chr1 | 69495 | 69495 | C | T | NA | NA | NA | NA | NA | NA | NA | NA | NA |
| chr1 | 69496 | 69496 | G | A | 0.0007 | 0.0061 | 0.0014 | 0 | 0 | 0 | 7.305e−05 | 0 | 8.709e−05 |
| chr1 | 69496 | 69496 | G | T | NA | NA | NA | NA | NA | NA | NA | NA | NA |
| chr1 | 69510 | 69510 | C | T | NA | NA | NA | NA | NA | NA | NA | NA | NA |
| chr1 | 69511 | 69511 | A | G | 0.9506 | 0.6074 | 0.9508 | 0.9779 | 0.9995 | 0.9915 | 0.9728 | 0.9499 | 0.9854 |
| chr1 | 69513 | 69513 | A | G | 2.544e−05 | 0 | 0 | 0 | 0.0002 | 0 | 1.489e−05 | 0 | 0 |
| chr1 | 69521 | 69521 | T | C | 3.801e−05 | 0.0002 | 0 | 0 | 0.0002 | 0 | 0 | 0 | 0 |
| chr1 | 69522 | 69522 | T | C | NA | NA | NA | NA | NA | NA | NA | NA | NA |
| chr1 | 69534 | 69534 | T | C | 0.0003 | 0 | 0 | 0 | 0.0032 | 0 | 0 | 0 | 0 |
| chr1 | 69543 | 69543 | C | A | NA | NA | NA | NA | NA | NA | NA | NA | NA |
| chr1 | 69550 | 69550 | G | A | NA | NA | NA | NA | NA | NA | NA | NA | NA |
| chr1 | 69550 | 69550 | G | T | NA | NA | NA | NA | NA | NA | NA | NA | NA |
| chr1 | 69552 | 69552 | G | A | 2.526e−05 | 8.604e−05 | 0 | 0 | 0 | 0 | 0 | 0 | 0.0001 |
| chr1 | 69555 | 69555 | T | G | 6.271e−06 | 8.603e−05 | 0 | 0 | 0 | 0 | 0 | 0 | 0 |
| chr1 | 69559 | 69559 | G | A | 9.668e−05 | 0 | 0 | 0 | 0.0010 | 0 | 0 | 0 | 0 |

The pLof sites were screened based on the above-mentioned annotation details. The screening step was as follows: only the variant sites corresponding to Stop-gained, Splicing, and Frameshift variant in the master transcript were remained, while other variant sites not present in the master transcript, but appearing in the non-main transcript were excluded

A Stop-gained variant referred to a variant where the original amino acids were changed to a termination codon, leading to premature termination of the protein transcript. A Splicing variant affected the normal transcription of the transcript, potentially resulting in the inclusion of intron region and causing variations in the protein structure. Frameshift variant occurred when there were alterations at exon-intron boundaries of RNA precursors and RNA adapter sites recognized by the spliceosome. Variant occurring in these regions could disrupt mRNA splicing and subsequently impact protein translation. Splicing was the process of removing introns and retaining exons during transcription. If a variant occurred at the classical splicing site, namely GT-AG variant, it was considered a pathogenic variant.

Following the screening, the remaining pLoF variant sites were shown below:

TABLE 7
pLoF List

| | Initiation | Termination | Reference | Variant | Corresponding | Variant | Variant | Confidence |
|---|---|---|---|---|---|---|---|---|
| Chromosome | position | position | base | base | gene | type | details | level |
| chr1 | 10002841 | 10002841 | C | G | LZIC | splicing | NM_032368: | LoF = |
| | | | | | | | exon2: | LC |
| | | | | | | | UTR5 | |
| chr1 | 100152230 | 100152230 | A | G | PALMD | splicing | NM_017734: | LoF = |
| | | | | | | | exon4: | HC |
| | | | | | | | c.252 − 2A > G | |
| chr1 | 100152248 | 100152248 | C | T | PALMD | stopgain | PALMD: | LoF = |
| | | | | | | | NM_017734: | HC |
| | | | | | | | exon4: | |
| | | | | | | | c.C268T: | |
| | | | | | | | p.Q90X | |
| chr1 | 100152747 | 100152747 | T | C | PALMD | splicing | NM_017734: | LoF = |
| | | | | | | | exon6: | HC |
| | | | | | | | c.514 + 2T > C | |
| chr1 | 100155308 | 100155308 | — | C | PALMD | frameshift | PALMD: | LoF = |
| | | | | | | insertion | NM_017734: | LC |
| | | | | | | | exon7: | |
| | | | | | | | c.1493dupC: | |
| | | | | | | | p.S498fs | |
| chr1 | 100155408 | 100155409 | CT | — | PALMD | frameshift | PALMD: | LoF = |
| | | | | | | deletion | NM_017734: | LC |
| | | | | | | | exon7: | |
| | | | | | | | c.1592_1593del: | |
| | | | | | | | p.T531fs | |
| chr1 | 100159602 | 100159602 | — | A | PALMD | frameshift | PALMD: | LoF = |
| | | | | | | insertion | NM_017734: | LC |
| | | | | | | | exon8: | |
| | | | | | | | c.1641dupA: | |
| | | | | | | | p.G547fs | |
| chr1 | 100174501 | 100174501 | C | A | FRRS1 | stopgain | FRRS1: | LoF = |
| | | | | | | | NM_001013660: | LC |
| | | | | | | | exon17: | |
| | | | | | | | c.G1834T: | |
| | | | | | | | p.E612X | |
| chr1 | 100174563 | 100174563 | — | G | FRRS1 | frameshift | FRRS1: | LoF = |
| | | | | | | insertion | NM_001013660: | LC |
| | | | | | | | exon17: | |
| | | | | | | | c.1771_1772insC: | |
| | | | | | | | p.F591fs | |
| chr1 | 100174614 | 100174614 | G | C | FRRS1 | stopgain | FRRS1: | LoF = |
| | | | | | | | NM_001013660: | LC |
| | | | | | | | exon17: | |
| | | | | | | | c.C1721G: | |
| | | | | | | | p.S574X | |
| chr1 | 10074675 | 100174675 | C | — | FRRS1 | frameshift | FRRS1: | LoF = |
| | | | | | | deletion | NM_001013660: | LC |
| | | | | | | | exon17: | |
| | | | | | | | c.1660delG: | |
| | | | | | | | p.V554fs | |
| chr1 | 100174677 | 100174677 | C | G | FRRS1 | splicing | NM_001013660: | LoF = |
| | | | | | | | exon17: | HC |
| | | | | | | | c.1659 − 1G > C | |
| chr1 | 100174767 | 100174767 | G | A | FRRS1 | stopgain | FRRS1: | LoF = |
| | | | | | | | NM_001013660: | LC |
| | | | | | | | exon16: | |
| | | | | | | | c.C1645T: | |
| | | | | | | | p.Q549X | |
| chr1 | 100176493 | 100176493 | A | — | FRRS1 | frameshift | FRRS1: | LoF = |
| | | | | | | deletion | NM_001013660: | HC |
| | | | | | | | exon15: | |
| | | | | | | | c.1493delT: | |
| | | | | | | | p.F498fs | |
| chr1 | 100185089 | 100185089 | C | T | FRRS1 | splicing | NM_001013660: | LoF = |
| | | | | | | | exon10: | HC |
| | | | | | | | c.1120 + 1G > A | |
| chr1 | 100185115 | 100185115 | — | T | FRRS1 | frameshift | FRRS1: | LoF = |
| | | | | | | insertion | NM_001013660: | HC |
| | | | | | | | exon10: | |
| | | | | | | | c.1094dupA: | |
| | | | | | | | p.H365fs | |
| chr1 | 100185172 | 100185172 | — | TCATTGGT | FRRS1 | frameshift | FRRS1: | LoF = |
| | | | | | | insertion | NM_001013660: | HC |
| | | | | | | | exon10: | |
| | | | | | | | c.1037_1038insACCAATGA: | |
| | | | | | | | p.L346fs | |
| chr1 | 100185177 | 100185177 | — | TAATCTCA | FRRS1 | stopgain | FRRS1: | LoF = |
| | | | | | | | NM_001013660: | HC |
| | | | | | | | exon10: | |
| | | | | | | | c.1032_1033insTGAGATTA: | |
| | | | | | | | p.P345_L346delinsX | |
| chr1 | 100194080 | 100194080 | G | T | FRRS1 | stopgain | FRRS1: | LoF = |
| | | | | | | | NM_001013660: | HC |
| | | | | | | | exon9: | |
| | | | | | | | c.C975A: | |
| | | | | | | | p.Y325X | |
| chr1 | 100195232 | 100195232 | G | A | FRRS1 | stopgain | FRRS1: | LoF = |
| | | | | | | | NM_001013660: | HC |
| | | | | | | | exon8: | |
| | | | | | | | c.C832T: | |
| | | | | | | | p.R278X | |
| chr1 | 100203683 | 100203683 | T | A | FRRS1 | stopgain | FRRS1: | LoF = |
| | | | | | | | NM_001013660: | HC |
| | | | | | | | exon7: | |
| | | | | | | | c.A718T: | |
| | | | | | | | p.K240X | |
| chr1 | 100203817 | 100203817 | — | CACT | FRRS1 | frameshift | FRRS1: | LoF = |
| | | | | | | insertion | NM_001013660: | HC |
| | | | | | | | exon7: | |
| | | | | | | | c.583_584inSAGTG: | |
| | | | | | | | p.A195fs | |
| chr1 | 100206498 | 100206498 | T | C | FRRS1 | splicing | NM_001013660: | LoF = |
| | | | | | | | exon6: | HC |
| | | | | | | | c.429 − 2A > G | |
| chr1 | 100207793 | 100207793 | T | — | FRRS1 | frameshift | FRRS1: | LoF = |
| | | | | | | deletion | NM_001013660: | HC |
| | | | | | | | exon5: | |
| | | | | | | | c.370delA: | |
| | | | | | | | p.T124fs | |
| chr1 | 100207799 | 100207799 | T | A | FRRS1 | stopgain | FRRS1: | LoF = |
| | | | | | | | NM_001013660: | HC |
| | | | | | | | exon5: | |
| | | | | | | | c.A364T: | |
| | | | | | | | p.K122X | |
| chr1 | 100212884 | 100212884 | — | CAAA | FRRS1 | stopgain | FRRS1: | LoF = |
| | | | | | | | NM_001013660: | HC |
| | | | | | | | exon4: | |
| | | | | | | | c.298_299inSTTTG: | |
| | | | | | | | p.E100_V101delinsVX | |
| chr1 | 100214128 | 100214128 | C | G | FRRS1 | splicing | NM_001013660: | LoF = |
| | | | | | | | exon3: | HC |
| | | | | | | | c.196 + 1G > C | |
| chr1 | 100214229 | 100214229 | G | T | FRRS1 | stopgain | FRRS1: | LoF = |
| | | | | | | | NM_001013660: | HC |
| | | | | | | | exon3: | |
| | | | | | | | c.C96A: | |
| | | | | | | | p.C32X | |
| chr1 | 100214236 | 100214237 | TG | — | FRRS1 | frameshift | FRRS1: | LoF = |
| | | | | | | deletion | NM_001013660: | HC |
| | | | | | | | exon3: | |
| | | | | | | | c.88_89del: | |
| | | | | | | | p.Q30fs | |
| chr1 | 100316682 | 100316682 | T | G | AGL | splicing | NM_000642: | LoF = |
| | | | | | | | exon2: | HC |
| | | | | | | | c.82 + 2T > G | |

Based on the obtained pLoF sites, Score3 was evaluated. For high-confidence pLoF variants, Score3 was assigned a value of +3, while for low-confidence pLoF variants, Score3 was assigned a value of +2.

### 2.4 Scoring the Variant Sites

Following the scoring of Score1 to Score3, the above variant sites were scored using the following formula:

$$Svar = {( Fq \times Pop \times 2 )}_{max = M} \times ( Score 1 + Score 2 + Score 3 )$$

wherein:
Svar represented a score of the variant site;
Fq represented a population variant frequency;
Pop represented a total population, which consisted of 42868 samples in the present Example;
M represented a pre-determined maximum value, set to 100 in the present Example.

Applying the formula allowed the calculation of the score of each variant site, Svar. If Svar exceeded 0, Svar was defined as 0.

### 3. Scoring a Region Sequence (S3)

### 3.1 Determination of Window Size

In a previous study, the inventors conducted an analysis using the dbSNP150, EXAC03 whole exome database, and genomeAD whole exome database. They examined the intervals between recorded variant sites in these databases and calculated the intervals between two adjacent variant sites. The analysis revealed that a window size of 25 bp could cover over 95% of the fragment intervals, as depicted in FIG. 2. Based on this finding, it was concluded that the window size should be set at 25 bp.

Consequently, the fragment was divided into a window size of 25 bp after each variant, resulting in a plurality of region sequences comprising variant sites.

### 3.2 Scoring the Sequences (Region Sequences)

Each region sequence was evaluated using the formula as follows:

$$S total = N \times {\sum }_{k = 0}^{n} Svar$$

wherein Stotal represented a total score of the region sequence, N represented a total number of variants in the region sequence.

### 4. Excluding Variant Sites (S4)

If the total score of the region sequence was lower than a predetermine value, a variant site within the region sequence was determined to be an artefact variant site and consequently excluded.

The excluding step comprised comparing the total scores (Stotal) of all region sequences either against a specific number (such as −100, or −50) or by identifying the top 5% of minimum scores when sorting the total scores of each region sequence from low to high. Any variant within the window that met the criteria was determined to be an artefact variant.

In the Example, a screen threshold value of −418 was set. If the Stotal was lower than −418 (i.e., within the top 5% minimum score when the total scores (Stotal) of each region sequence was sorted from low to high), the region sequence was flagged as a positive result, indicating the presence of an artefact variant. The table below illustrated some artefact variants, displaying those with high negative scores or low negative scores.

TABLE 8
Artefact variants and the corresponding windows,
total variants in the windows, and total scores

| | | | Total variants | |
|---|---|---|---|---|
| Chromosome | Position | Window | in the window | Total score |
| chr9 | 35906583 | chr9:35906583-35906607 | 428 | −7026708.30830639 |
| chr9 | 35906584 | chr9:35906584-35906608 | 415 | −6726549.28627591 |
| chr9 | 35906585 | chr9:35906585-35906609 | 414 | −6704131.02405082 |
| chr9 | 35906586 | chr9:35906586-35906610 | 413 | −6665636.51330758 |
| chr9 | 35906580 | chr9:35906580-35906604 | 401 | −6078580.34869512 |
| chr9 | 35906582 | chr9:35906582-35906606 | 398 | 5996092.36077493 |
| chr9 | 35906581 | chr9:35906581-35906605 | 398 | 5995694.37824108 |
| chr9 | 35906577 | chr9:35906577-35906601 | 388 | 5743785.43456742 |
| chrX | 100603410 | chrX:100603410-100603434 | 171 | 5728052.73344433 |
| chrX | 100603413 | chrX:100603413-100603437 | 170 | −5694215.36395906 |
| chr9 | 35906587 | chr9:35906587-35906611 | 350 | −4800809.36996248 |
| chr9 | 35906589 | chr9:35906589-35906613 | 347 | −4665279.90481323 |
| chr9 | 35906588 | chr9:35906588-35906612 | 342 | −4574801.96584414 |
| chr9 | 35906590 | chr9:35906590-35906614 | 341 | 4569608.93720737 |
| chr9 | 35906592 | chr9:35906592-35906616 | 344 | 4437122.81440532 |
| chr9 | 35906591 | chr9:35906591-35906615 | 340 | 4387228.27972579 |
| chr9 | 35906576 | chr9:35906576-35906600 | 309 | 3402299.57000941 |
| chr2 | 11727844 | chr2:11727844-11727868 | 126 | −3396924.90964996 |
| chr2 | 11727843 | chr2:11727843-11727867 | 123 | 3379268.24098448 |
| chr9 | 35906575 | chr9:35906575-35906599 | 305 | −3347277.37297846 |
| chr2 | 11727854 | chr2:11727854-11727878 | 134 | 3326648.77447192 |
| chr2 | 11727846 | chr2:11727846-11727870 | 134 | 3326380.78853077 |
| chr18 | 77136193 | chr18:77136193-77136217 | 133 | −3311664.8494566 |
| chr2 | 11727847 | chr2:11727847-11727871 | 133 | 3301291.06297629 |
| chr2 | 11727856 | chr2:11727856-11727880 | 132 | 3275413.37200303 |
| chr18 | 77136195 | chr18:77136195-77136219 | 134 | 3257772.50770687 |
| chr2 | 11727857 | chr2:11727857-11727881 | 131 | 3249813.66882979 |
| chr9 | 35906574 | chr9:35906574-35906598 | 298 | −3237378.0933243 |
| chr2 | 11727858 | chr2:11727858-11727882 | 130 | 3224225.96512994 |
| chr2 | 11727861 | chr2:11727861-11727885 | 129 | 3215677.48708922 |
| chr18 | 77136197 | chr18:77136197-77136221 | 134 | 3178176.47012576 |
| chr2 | 11727863 | chr2:11727863-11727887 | 127 | 3162266.19129363 |
| chr9 | 35906571 | chr9:35906571-35906595 | 290 | −3121469.8819954 |
| chr18 | 77136198 | chr18:77136198-77136222 | 133 | 3074658.73527408 |
| chr9 | 70871928 | chr9:70871928-70871952 | 193 | 3048926.13846847 |
| chr9 | 70871929 | chr9:70871929-70871953 | 192 | 3031976.64755712 |
| chr18 | 77136191 | chr18:77136191-77136215 | 124 | 3012426.70434945 |
| chr9 | 70871930 | chr9:70871930-70871954 | 190 | −2994693.82716957 |
| chr2 | 11727864 | chr2:11727864-11727888 | 123 | −2979273.69614749 |
| chr9 | 70871931 | chr9:70871931-70871955 | 188 | −2960162.87270482 |
| chr2 | 11727865 | chr2:11727865-11727889 | 122 | −2954807.96948833 |
| chr5 | 167945074 | chr5:167945074-167945098 | 200 | 2930730.88303328 |
| chr18 | 77136190 | chr18:77136190-77136214 | 118 | 2861236.37222784 |
| chr2 | 11727867 | chr2:11727867-11727891 | 117 | 2708755.82961834 |
| chr11 | 117039153 | chr11:117039153-117039177 | 140 | 2604220.84949085 |
| chr1 | 1247578 | chr1:1247578-1247602 | 280 | 2601686.59648442 |
| chr1 | 1247579 | chr1:1247579-1247603 | 279 | −2587373.0933815 |
| chr11 | 117039151 | chr11:117039151-117039175 | 139 | 2584785.31097787 |
| chr11 | 117039154 | chr11:117039154-117039178 | 139 | 2584785.31097787 |
| chr1 | 1247574 | chr1: 1247574-1247598 | 278 | −2582547.14519519 |
| chr9 | 32986030 | chr9:32986030-32986054 | 45 | −4409.795665296 |

Note: Each variant site corresponded to at least one window starting from its own position, but it might also fall within other windows. If a site was found in a window identified as having an artefact variant, even if it also fell within other windows that were not considered to have an artefact variant, the site was still determined to be an artefact variant site.

Based on the above results, a total of 155,338 artefact variants were identified out of 8,245,702 variants in the present Example, resulting in a screening rate of 1.88%. This meant that 8,090,364 sites, accounting for 98.12% of the total remained within the normal screening scope (more than 95%).

### 5. Validation

Two window fragments that were identified as having artefact variants were selected for verification.

### 5.1 Fragment 1

Fragment 1 corresponded to a window size from chr9:35906583 to 35906607, specifically Chr 9: 35906583 site. Some variants within the window were shown below.

TABLE 9
Variants of the window (chr9: 35906583-35906607) corresponded to
chr9: 35906583 site

| | Initiation | Termination | Reference | |
|---|---|---|---|---|
| Chromone | position | position | base | Variant base |
| chr9 | 35906583 | 35906583 | — | SEQ ID NO: 1 (CCACCACCACCA) |
| chr9 | 35906583 | 35906583 | — | SEQ ID NO: 2 |
| | | | | (CCACCACCACCACCACCCCCACCACCA |
| | | | | CCCCCACCACACCCCTCACCACCTCCAC |
| | | | | CACCACCACCACCCCCACCACCACCCCC |
| | | | | GCCACACCCCTCACCACCT) |
| chr9 | 35906583 | 35906583 | — | SEQ ID NO: 3 |
| | | | | (CCACCACCACCACCACCCCCACCACCA |
| | | | | CCCCCGCCACACCCCTCACCACCT) |
| chr9 | 35906583 | 35906583 | — | SEQ ID NO: 4 |
| | | | | (CCACCACCACCACCACCCCCACCCCCG |
| | | | | CCACACCCCTCACCACCT) |
| chr9 | 35906583 | 35906583 | — | SEQ ID NO: 5 |
| | | | | (CCACCACCACCACCCCCACCCCCGCCA |
| | | | | CACCCCTCACCACCT) |
| chr9 | 35906583 | 35906583 | — | SEQ ID NO: 6 |
| | | | | (CCACCACCACCACCCCCACCCCCGCCA |
| | | | | CACCCCTCACCACCTCCACCACCACCAC |
| | | | | CCCCACCCCCGCCACACCCCTCACCACC |
| | | | | T) |
| chr9 | 35906583 | 35906583 | — | SEQ ID NO: 7 |
| | | | | (CCACCACCTCCACCACCACCACCACCA |
| | | | | CCCCCGCCACACCCCTCACCA) |
| chr9 | 35906583 | 35906583 | — | SEQ ID NO: 8 |
| | | | | (CCACCACCTCCACCACCACCACCACCA |
| | | | | CCCCCGCCACACCCCTCACCACCTCCAC |
| | | | | CA) |
| chr9 | 35906583 | 35906583 | — | SEQ ID NO: 9 (CCACCTCCACCA) |
| chr9 | 35906583 | 35906583 | — | SEQ ID NO: 10 |
| | | | | (CCACCTCCACCACCACCA) |
| chr9 | 35906583 | 35906583 | — | SEQ ID NO: 11 (CCACTACCACCA) |
| chr9 | 35906583 | 35906583 | | CCCA |
| chr9 | 35906583 | 35906583 | — | SEQ ID NO: 12 |
| | | | | (CCCCGCCACACCCCTCACCACCTCCACC |
| | | | | ACCACCACCCCCGCCACACCCCTCACCA |
| | | | | CCTCCACCACCACCA) |
| chr9 | 35906583 | 35906583 | — | SEQ ID NO: 13 (CTACCACCACCA) |
| chr9 | 35906583 | 35906583 | T | — |
| chr9 | 35906583 | 35906583 | T | A |
| chr9 | 35906583 | 35906583 | T | C |
| chr9 | 35906584 | 35906592 | CCACCACCA | — |
| chr9 | 35906585 | 35906595 | SEQ ID NO: | — |
| | | | 14 | |
| | | | (CACCACCA | |
| | | | CCA) | |
| chr9 | 35906586 | 35906586 | — | SEQ ID NO: 15 |
| | | | | (CCACCACCACCACCACCACCACCACAC |
| | | | | CCCTCACCACCT) |
| chr9 | 35906586 | 35906586 | — | SEQ ID NO: 16 |
| | | | | (CCACCACCACCACCACCACCACCACCA |
| | | | | TCCCCGCCACACCCCTCACCACCT) |
| chr9 | 35906586 | 35906586 | — | SEQ ID NO: 17 |
| | | | | (CCACCACCACCACCACCACCACCACCC |
| | | | | CCACCACACCCCTCACCACCT) |
| chr9 | 35906586 | 35906586 | — | SEQ ID NO: 18 |
| | | | | (CCACCACCACCACCACCACCACCACCC |
| | | | | CCACCACCT) |
| chr9 | 35906586 | 35906586 | — | SEQ ID NO: 19 |
| | | | | (CCACCACCACCACCACCACCACCACCC |
| | | | | CCACCCCCACCGCCACCATCCCCGCCAC |
| | | | | ACCCCTCACCACCT) |
| chr9 | 35906586 | 35906586 | — | SEQ ID NO: 20 |
| | | | | (CCACCACCACCACCACCACCACCACCC |
| | | | | CCACCCCCGCCACACCCCTCACCACCT) |
| chr9 | 35906586 | 35906586 | — | SEQ ID NO: 21 |
| | | | | (CCACCACCACCACCACCACCACCACCC |
| | | | | CCACCGCCACCATCCCCGCCACACCCCT |
| | | | | CACCACCT) |
| chr9 | 35906586 | 35906586 | — | SEQ ID NO: 22 |
| | | | | (CCACCACCACCACCACCACCACCACCC |
| | | | | CCACCGCCACCATCCCCGCCACACCCCT |
| | | | | CACCACCTCCACCACCACCACCACCACC |
| | | | | CCCGCCACACCCCTCACCACCT) |
| chr9 | 35906586 | 35906586 | — | SEQ ID NO: 23 |
| | | | | (CCACCACCACCACCACCACCACCACCC |
| | | | | CCGCCACACCCCTCACCACCT) |
| chr9 | 35906586 | 35906586 | — | SEQ ID NO: 24 |
| | | | | (CCACCACCACCACCACCACCACCATCC |
| | | | | CCGCCACGCCCCTCACCACCT) |
| chr9 | 35906586 | 35906586 | — | SEQ ID NO: 25 |
| | | | | (CCACCACCACCACCACCACCACCCCCG |
| | | | | CCACACCCCTCACCACCTCCACCACCAC |
| | | | | CACCACCACCCCCGCCACACCCCTCACC |
| | | | | ACCT) |
| chr9 | 35906586 | 35906586 | — | SEQ ID NO: 26 |
| | | | | (CCACCACCACCACCACCACCCCCACCC |
| | | | | CCACCGCCACCACCCCCGCCACACCCCT |
| | | | | CACCACCT) |
| chr9 | 35906586 | 35906586 | — | SEQ ID NO: 27 |
| | | | | (CCACCACCACCACCACCACCCCCACCC |
| | | | | CCACCGCCACCATCCCCGCCACACCCCT |
| | | | | CACCACCT) |
| chr9 | 35906586 | 35906586 | — | SEQ ID NO: 28 |
| | | | | (CCACCACCACCACCACCACCCCCACCG |
| | | | | CCACACCCCTCACCACCT) |
| chr9 | 35906586 | 35906586 | — | SEQ ID NO: 29 |
| | | | | (CCACCACCACCACCACCACCCCCACCG |
| | | | | CCACCATCCCCGCCACACCCCTCACCAC |
| | | | | CT) |
| chr9 | 35906586 | 35906586 | — | SEQ ID NO: 30 |
| | | | | (CCACCACCACCACCACCACCCCCGCCA |
| | | | | CACCCCTCACCACCT) |
| chr9 | 35906586 | 35906586 | — | SEQ ID NO: 31 |
| | | | | (CCACCACCACCACCACCACCGCCACAC |
| | | | | CCCTCACCACCT) |
| chr9 | 35906586 | 35906586 | — | SEQ ID NO: 32 |
| | | | | (CCACCACCACCACCACCACCGCCACCA |
| | | | | TCCCCGCCACACCCCTCACCACCT) |
| chr9 | 35906586 | 35906586 | — | SEQ ID NO: 33 |
| | | | | (CCACCACCACCACCACCCCCACCACAC |
| | | | | CCCTCACCACCT) |
| chr9 | 35906586 | 35906586 | — | SEQ ID NO: 34 |
| | | | | (CCACCACCACCACCACCCCCACCATCCC |
| | | | | CGCCACACCCCTCACCACCTCCACCACC |
| | | | | ACCACCACCACCCCCGCCACACCCCTCA |
| | | | | CCACCT) |
| chr9 | 35906586 | 35906586 | — | SEQ ID NO: 35 |
| | | | | (CCACCACCACCACCACCCCCACCCCCA |
| | | | | CCGCCACCATCCCCGCCACACCCCTCAC |
| | | | | CACCT) |
| chr9 | 35906586 | 35906586 | — | SEQ ID NO: 36 |
| | | | | (CCACCACCACCACCACCCCCGCCACAC |
| | | | | CCCTCACCACCT) |
| chr9 | 35906586 | 35906586 | — | SEQ ID NO: 37 |
| | | | | (CCACCACCACCACCACCCCCGCCACAC |
| | | | | CCCTCACCACCTCCACCACCACCACCAC |
| | | | | CACCCCCGCCACACCCCTCACCACCT) |
| chr9 | 35906586 | 35906586 | — | SEQ ID NO: 38 |
| | | | | (CCACCACCACCACCACCCCCGCCACAC |
| | | | | CCCTCACCACCTCCACCACCACCACCAC |
| | | | | CCCCGCCACACCCCTCACCACCT) |
| chr9 | 35906586 | 35906586 | — | SEQ ID NO: 39 |
| | | | | (CCACCACCACCACCACCCCCGCCACAC |
| | | | | CCCTCACCACCTCCACCACCACCACCC) |
| chr9 | 35906586 | 35906586 | — | SEQ ID NO: 40 |
| | | | | (CCACCACCACCACCACCCCCGCCACAC |
| | | | | CCCTCACCACCTCCACCACCACCACCCC |
| | | | | CACCCCCGCCACACCCCTCACCACCT) |
| chr9 | 35906586 | 35906586 | — | SEQ ID NO: 41 |
| | | | | (CCACCACCACCACCACCCCCGCCACAC |
| | | | | CCCTCACCACCTCCACCACCACCACCCC |
| | | | | CACCGCCACCATCCCCGCCACACCCCTC |
| | | | | ACCACCT) |
| chr9 | 35906586 | 35906586 | — | SEQ ID NO: 42 |
| | | | | (CCACCACCACCACCACCCCCGCCACAC |
| | | | | CCCTCACCACCTCCACCACCACCACCCC |
| | | | | CGCCACACCCCTCACCACCT) |
| chr9 | 35906586 | 35906586 | — | SEQ ID NO: 43 |
| | | | | (CCACCACCACCACCACCCCCGCCACAC |
| | | | | CCCTCACCCCCT) |
| chr9 | 35906586 | 35906586 | — | SEQ ID NO: 44 |
| | | | | (CCACCACCACCACCACCCCCGCCACCA |
| | | | | TCCCCGCCACACCCCTCACCACCT) |
| chr9 | 35906586 | 35906586 | — | SEQ ID NO: 45 |
| | | | | (CCACCACCACCACCACCCCCGCCACCA |
| | | | | TCCCCGCCACACCCCTCACCACCTCCAC |
| | | | | CACCACCACCACCACCCCCGCCACACCC |
| | | | | CTCACCACCT) |
| chr9 | 35906586 | 35906586 | — | SEQ ID NO: 46 |
| | | | | (CCACCACCACCACCACCGCCACCATCC |
| | | | | CCGCCACACCCCTCACCACCT) |
| chr9 | 35906586 | 35906586 | | SEQ ID NO: 47 (CCACCACCACCACCC) |
| chr9 | 35906586 | 35906586 | — | SEQ ID NO: 48 |
| | | | | (CCACCACCACCACCCCCACCACACCCCT |
| | | | | CACCACCT) |
| chr9 | 35906586 | 35906586 | — | SEQ ID NO: 49 |
| | | | | (CCACCACCACCACCCCCACCACCACCA |
| | | | | CCCCCACCGCCACCATCCCCGCCACACC |
| | | | | CCTCACCACCT) |
| chr9 | 35906586 | 35906586 | — | SEQ ID NO: 50 |
| | | | | (CCACCACCACCACCCCCACCACCACCA |
| | | | | CCCCCGCCACACCCCTCACCACCT) |
| chr9 | 35906586 | 35906586 | — | SEQ ID NO: 51 |
| | | | | (CCACCACCACCACCCCCACCACCACCC |
| | | | | CCACCACACCCCTCACCACCT) |
| chr9 | 35906586 | 35906586 | — | SEQ ID NO: 52 |
| | | | | (CCACCACCACCACCCCCACCACCACCC |
| | | | | CCACCACCCCCGCCACACCCCTCACCAC |
| | | | | CT) |
| chr9 | 35906586 | 35906586 | — | SEQ ID NO: 53 |
| | | | | (CCACCACCACCACCCCCACCACCACCC |
| | | | | CCGCCACACCCCTCACCACCT) |
| chr9 | 35906586 | 35906586 | — | SEQ ID NO: 54 |
| | | | | (CCACCACCACCACCCCCCCCGCCACAC |
| | | | | CCCTCACCACCT) |
| chr9 | 35906586 | 35906586 | — | SEQ ID NO: 55 |
| | | | | (CCACCACCACCACCCCCGCCACACCCCT |
| | | | | CACCACCT) |
| chr9 | 35906586 | 35906586 | — | SEQ ID NO: 56 |
| | | | | (CCACCACCACCACCCCCGCCACACCCCT |
| | | | | CACCACCTCCACCACCACCACCACCACC |
| | | | | CCCGCCACACCCCTCACCACCT) |
| chr9 | 35906586 | 35906586 | — | SEQ ID NO: 57 |
| | | | | (CCACCACCACCACCCCCGCCACCCCCCC |
| | | | | CACCACCTCCACCCCCACCACCACCACC |
| | | | | CCCGCCACACCCCTCACCACCTCCACCA |
| | | | | CCACCACCACCACCCCCGCCACACCCCT |
| | | | | CACCACCT) |
| chr9 | 35906586 | 35906586 | — | SEQ ID NO: 58 |
| | | | | (CCACCACCACCACCTCCACCACCACCA |
| | | | | CCACCCCCACCGCCACCATCCCCGCCAC |
| | | | | ACCCCTCACCACCT) |
| chr9 | 35906586 | 35906586 | — | SEQ ID NO: 59 (CCACCACCACCC) |
| chr9 | 35906586 | 35906586 | — | SEQ ID NO: 60 |
| | | | | (CCACCACCACCCCCACCACCACCCCCA |
| | | | | CCACACCCCTCACCACCT) |
| chr9 | 35906586 | 35906586 | — | SEQ ID NO: 61 |
| | | | | (CCACCACCACCCCCACCCCCGCCACAC |
| | | | | CCCTCACCACCT) |
| chr9 | 35906586 | 35906586 | — | SEQ ID NO: 62 |
| | | | | (CCACCACCACCCCCACCCCCGCCACAC |
| | | | | CCCTCACCACCTCCACCACCACCACCAC |
| | | | | CACCACCGCCACACCCCTCACCACCT) |
| chr9 | 35906586 | 35906586 | — | SEQ ID NO: 63 |
| | | | | (CCACCACCACCCCCACCCCCGCCACAC |
| | | | | CCCTCACCACCTCCACCACCACCACCAC |
| | | | | CACCCCCGCCACACCCCTCACCACCT) |
| chr9 | 35906586 | 35906586 | — | SEQ ID NO: 64 |
| | | | | (CCACCACCACCCCCACCG) |
| chr9 | 35906586 | 35906586 | — | SEQ ID NO: 65 |
| | | | | (CCACCACCACCCCCACCGCCACACCCCT |
| | | | | CACCACCTCCACCACCACCACCACCACC |
| | | | | CCCGCCACACCCCTCACCACCT) |
| chr9 | 35906586 | 35906586 | — | SEQ ID NO: 66 |
| | | | | (CCACCACCACCCCCACCGCCACCACCC |
| | | | | CCGCCACACCCCTCACCACCT) |
| chr9 | 35906586 | 35906586 | — | SEQ ID NO: 67 |
| | | | | (CCACCACCACCCCCACCGCCACCATCCC |
| | | | | CGCCACACCCCTCACCACCT) |
| chr9 | 35906586 | 35906586 | — | SEQ ID NO: 68 |
| | | | | (CCACCACCACCCCCACCGCCACCATCCC |
| | | | | CGCCACACCCCTCACCACCTCCACCACC |
| | | | | ACCACCACCACCCCCGCCACACCCCTCA |
| | | | | CCACCT) |
| chr9 | 35906586 | 35906586 | — | SEQ ID NO: 69 |
| | | | | (CCACCACCACCCCCACCGCCACCATCCC |
| | | | | CGCCACACCCCTCACCACCTCCACCACC |
| | | | | GCCACCATCCCCGCCACACCCCTCACCA |
| | | | | CCT) |
| chr9 | 35906586 | 35906586 | — | SEQ ID NO: 70 |
| | | | | (CCACCACCACCCCCACCGCCACCATCCC |
| | | | | CGCCACGCCCCTCACCACCT) |
| chr9 | 35906586 | 35906586 | — | SEQ ID NO: 71 |
| | | | | (CCACCACCACCCCCGCCACACCCCTCAC |
| | | | | CACCT) |
| chr9 | 35906586 | 35906586 | — | SEQ ID NO: 72 (CCACCACCACCT) |
| chr9 | 35906586 | 35906586 | — | SEQ ID NO: 73 |
| | | | | (CCACCACCCCCACCCCCACCGCCACCAT |
| | | | | CCCCGCCACACCCCTCACCACCT) |
| chr9 | 35906586 | 35906586 | — | SEQ ID NO: 74 |
| | | | | (CCACCACCCCCGCCACACCCCTCACCAC |
| | | | | CTCCACCACCACCACCACCACCCCCGCC |
| | | | | ACACCCCTCACCACCT) |
| chr9 | 35906586 | 35906586 | — | SEQ ID NO: 75 |
| | | | | (CCACCACCGCCACCATCCCCGCCACAC |
| | | | | CCCTCACCACCT) |
| chr9 | 35906586 | 35906586 | — | SEQ ID NO: 76 |
| | | | | (CCACCACCTCCACCACCT) |
| chr9 | 35906586 | 35906586 | — | SEQ ID NO: 77 |
| | | | | (CCACCCCCACCACCACCCCCGCCACAC |
| | | | | CCCTCACCACCT) |
| chr9 | 35906586 | 35906586 | — | CCACCT |
| chr9 | 35906586 | 35906586 | — | SEQ ID NO: 78 |
| | | | | (CCACCTCCACCACCACCACCACCACCCC |
| | | | | CGCCACACCCCTCACCACCT) |
| chr9 | 35906586 | 35906586 | — | SEQ ID NO: 79 |
| | | | | (CCACCTCCACCCCCACCACCACCACCCC |
| | | | | CGCCACACCCCTCACCACCTCCACCACC |
| | | | | TCCACCACCACCACCACCACCCCCGCCA |
| | | | | CACCCCTCACCACCT) |
| chr9 | 35906586 | 35906586 | — | CCC |
| chr9 | 35906586 | 35906586 | — | CCT |
| chr9 | 35906586 | 35906586 | A | C |
| chr9 | 35906586 | 35906586 | A | T |
| chr9 | 35906587 | 35906587 | — | SEQ ID NO: 80 |
| | | | | (CACCACCACCACCACCACCACCACCCC |
| | | | | CACCACACCCCTCACCACCTCCACCACC |
| | | | | ACCTCCACCACCTCCAC) |
| chr9 | 35906587 | 35906587 | — | SEQ ID NO: 81 |
| | | | | (CACCACCACCACCACCCCCGCCACACC |
| | | | | CCT) |
| chr9 | 35906587 | 35906587 | — | SEQ ID NO: 82 |
| | | | | (CACCACCACCACCTCCAC) |
| chr9 | 35906587 | 35906587 | — | SEQ ID NO: 83 (CACCACCTCCAC) |
| chr9 | 35906587 | 35906587 | — | SEQ ID NO: 84 |
| | | | | (CACCACCTCCACCACCTCCAC) |
| chr9 | 35906587 | 35906587 | — | SEQ ID NO: 85 |
| | | | | (CACCACCTCCACCCCCACCACCACCCCC |
| | | | | ACCACACCCCTCACCACCTCCACCACCA |
| | | | | CCTCCAC) |
| chr9 | 35906587 | 35906592 | CCACCA | — |
| chr9 | 35906587 | 35906607 | SEQ ID NO: | — |
| | | | 86 | |
| | | | (CCACCACC | |
| | | | ACCACCCCC | |
| | | | ACCG) | |
| chr9 | 35906588 | 35906588 | — | SEQ ID NO: 87 (ACCACCACCACC) |
| chr9 | 35906589 | 35906589 | — | SEQ ID NO: 88 |
| | | | | (CCACCACCACCACCCCCGCCACACCCCT |
| | | | | CACCACCTCCACCACCACCACCACCC) |
| chr9 | 35906589 | 35906589 | — | SEQ ID NO: 89 |
| | | | | (CCACCACCACCACCCCCGCCACACCCCT |
| | | | | CACCACCTCCC) |
| chr9 | 35906589 | 35906589 | — | CCACCACCC |
| chr9 | 35906589 | 35906589 | — | CCACCC |
| chr9 | 35906589 | 35906589 | — | CCACCT |
| chr9 | 35906589 | 35906589 | — | CCT |
| chr9 | 35906589 | 35906589 | A | — |
| chr9 | 35906589 | 35906589 | A | C |

Within the window (chr9:35906583-35906607), there were a total of 428 variant sites (a subset of these sites was exemplified in the table as 35906583-35906589). According to information from dbSNP, ClinVar and gnomAD-exome databases, windows with a length of 25 bp typically comprised fewer than 18 variant sites in 95% of cases. However, in this particular window (chr9:35906583-35906607), more than 18 variant sites were observed, indicating the presence of artefact variants. Furthermore, most of these artefact variants were INDELs, with only a few being SNVs. The occurrence of such a high number of INDELs in the same region significantly reduces the accuracy of INDELs classification. FIG. 3 showed the visualization results of IGV for the window comprising the site, comparing the reads of this region between two samples with a window length of 25 bp. The analysis reveals poor quality in this region, characterized by high GC content and inadequate confidence. Therefore, based on the evidence, the variants in this region were determined to be artefact variants, aligning with the conclusions derived from the method of the present disclosure.

### 5.5 Fragment 2

Fragment 2 corresponded to a window from chr9:32986030 to 32986054, specifically chr9: 32986030 site. The table below and FIG. 4 showed some variants within this window.

TABLE 10
Variants of the window (chr9: 32986030-32986054) corresponded
to chr9: 32986030 site

| chromo- | Initiation | Termination | Reference | |
|---|---|---|---|---|
| some | position | position | base | Variant base |
| chr9 | 32986030 | 32986030 | — | A |
| chr9 | 32986030 | 32986030 | — | SEQ ID NO: 90 |
| | | | | (AAAAAAAAACA |
| | | | | A) |
| chr9 | 32986030 | 32986030 | T | — |
| chr9 | 32986030 | 32986030 | T | A |
| chr9 | 32986030 | 32986030 | T | C |
| chr9 | 32986031 | 32986031 | A | — |
| chr9 | 32986031 | 32986032 | AA | — |
| chr9 | 32986031 | 32986043 | SEQ ID NO: 91 | — |
| | | | (AAAAAAAAAA | |
| | | | CAA) | |
| chr9 | 32986031 | 32986053 | SEQ ID NO: 92 | — |
| | | | (AAAAAAAAAACAA | |
| | | | AAAAAAAAAC) | |
| chr9 | 32986032 | 32986053 | SEQ ID NO: 93 | — |
| | | | (AAAAAAAAACAAA | |
| | | | AAAAAAAAC) | |
| chr9 | 32986033 | 32986053 | SEQ ID NO: 94 | — |
| | | | (AAAAAAAACAAAA | |
| | | | AAAAAAAC) | |
| chr9 | 32986034 | 32986053 | SEQ ID NO: 95 | — |
| | | | (AAAAAAACAAAAA | |
| | | | AAAAAAC) | |
| chr9 | 32986035 | 32986053 | SEQ ID NO: 96 | — |
| | | | (AAAAAACAAAAAA | |
| | | | AAAAAC) | |
| chr9 | 32986036 | 32986053 | SEQ ID NO: 97 | — |
| | | | (AAAAACAAAAAAA | |
| | | | AAAAC) | |
| chr9 | 32986037 | 32986037 | — | AAC |
| chr9 | 32986038 | 32986038 | — | AC |
| chr9 | 32986038 | 32986053 | SEQ ID NO: 98 | — |
| | | | (AAACAAAAAAAAA | |
| | | | AAC) | |
| chr9 | 32986039 | 32986039 | — | C |
| chr9 | 32986039 | 32986039 | A | C |
| chr9 | 32986040 | 32986040 | A | C |
| chr9 | 32986041 | 32986041 | — | A |
| chr9 | 32986041 | 32986041 | C | — |
| chr9 | 32986041 | 32986041 | C | A |
| chr9 | 32986042 | 32986056 | SEQ ID NO: 99 | — |
| | | | (AAAAAAAAAAACA | |
| | | | AA) | |
| chr9 | 32986044 | 32986044 | — | C |
| chr9 | 32986044 | 32986056 | SEQ ID NO: 100 | — |
| | | | (AAAAAAAAACAAA) | |
| chr9 | 32986045 | 32986045 | — | C |
| chr9 | 32986045 | 32986045 | A | C |
| chr9 | 32986046 | 32986046 | — | C |
| chr9 | 32986046 | 32986046 | A | C |
| chr9 | 32986047 | 32986047 | — | C |
| chr9 | 32986047 | 32986047 | A | C |
| chr9 | 32986048 | 32986048 | — | C |
| chr9 | 32986048 | 32986048 | A | C |
| chr9 | 32986049 | 32986049 | — | C |
| chr9 | 32986049 | 32986049 | A | C |
| chr9 | 32986050 | 32986050 | — | C |
| chr9 | 32986050 | 32986050 | A | T |
| chr9 | 32986052 | 32986052 | — | AC |
| chr9 | 32986052 | 32986053 | AC | — |
| chr9 | 32986053 | 32986053 | C | — |
| chr9 | 32986053 | 32986053 | C | A |
| chr9 | 32986054 | 32986054 | — | C |
| chr9 | 32986054 | 32986054 | A | C |
| chr9 | 32986054 | 32986057 | AAAA | — |

In this window (chr9:32986030-32986054), a total of 45 variant sites were identified. According to data from dbSNP, ClinVar and gnomAD-exome databases, t windows with a length of 25 bp comprised fewer than 18 variant sties in 95% of cases. However, in this particular window (chr9:32986030-32986054), there were more than 18 variant sites. These variant sites were scattered and located in proximity to each other, exhibiting pronounced ploy-A features that were prone to errors. FIG. 4 illustrates the visualization result of IGV for this site, comparing the reads of this region between 2 samples with a window length of 25 bp. The analysis revealed poor quality in this region and inadequate confidence. Therefore, based on the evidence, the variants within this region were determined to be artefact variants, aligning with the conclusions derived from the method of the present disclosure.

The validation results confirm that the method of the present disclosure can exclude most artefact variants, retaining a substantial number of true variants.

Although the present invention has been disclosed in the form of embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A method of reducing an artefact variant in high-throughput sequencing, comprising steps of:
a) building a population variant frequency database comprising: collecting high-throughput sequencing data from a plurality of samples and integrating to obtain a population variant frequency-Fq at each variant site, along with total population-Pop;
b) scoring a variant site comprising: obtaining high-throughput sequencing data from samples to be tested, then merging information from each variant site, and scoring each variant site using the following formula:
$$Svar = {( Fq \times Pop \times 2 )}_{max = M} \times ( Score 1 + Score 2 + Score 3 )$$
wherein Svar represents a score of the variant site, Fq represents a population variant frequency, Pop represents a total population, M represents a pre-determined maximum value, Score1 represents a variant type score, Score2 represents a variant coordinate score, Score3 represents a pLoF score;
wherein,
Score1 is evaluated based on the following standard: if a variant is known, Score1 is assigned a value of 0; if the variant is unknown, Score1 is assigned a negative value;
Score2 is evaluated based on the following standard: if the variant is unknown, Score2 is assigned a negative value according to a pre-set rule based on a location of the variant within a refGene region;
Score3 is evaluated based on the following standard: Score3 is assigned a positive value based on a predefined rule that determines a confidence level of predicted loss-of-function (LOF) variants filtered using LOFTEE;
c) scoring region sequence comprising: dividing a sequence into a predetermined window size after each variant to obtain a plurality of region sequences comprising variant sites,
wherein each region sequence is evaluated according to following formula:
$$S total = N \times {\sum }_{k = 0}^{n} Svar$$
wherein, Stotal represents a total score of the region sequence, N represents a total number of variants within the region sequence; and
d) excluding a variant site: if the total score of the region sequence Stotal is lower than a predetermined threshold value, a variant site within the region sequence is determined to be an artefact variant site and is excluded.

2. The method of reducing an artefact variant in high-throughput sequencing of claim 1, wherein in the step of building a population variant frequency database, each variant site is sorted based on chromosome and genome position.

3. The method of reducing an artefact variant in high-throughput sequencing of claim 1, wherein in the step of scoring a variant site, Score1 is evaluated based on the following standard: if a variant site has an annotation in a database selected from a group consisting of dbSNP, ClinVar, and geomAD-exome, the variant site is determined to be known and Score1 is assigned a value of zero; and if the variant site lacks such annotation, the variant site is determined to be unknown, and Score1 is assigned a negative value;
Score2 is evaluated based on the following standard: if the variant is unknown and falls within a region selected from a group consisting of exonic region, splicing region, UTR-3 region, UTR-5 region, upstream region, downstream region, intronic region, intergenic region, and ncRNA region in refGene, Score2 is assigned a negative value according to the pre-set rule; and
Score3 is evaluated based on the following standard: if the variant is determined to be a high-confidence or low-confidence predicted loss-of-function (pLoF) variant filtered using LOFTEE, Score3 is assigned a weighted value determined by a predetermined rule.

4. The method of reducing an artefact variant in high-throughput sequencing of claim 3, wherein in the step of scoring a variant site,
Score1 is evaluated based on the following standard: if the variant is known, Score1 is assigned a value of 0; if the variant is an unknown single nucleotide variant, Score1 is assigned a value of −1; if the variant is unknown insertion-deletion variant, Score1 is assigned a value of −5;
Score2 is evaluated based on the following standard: if the variant is located in an exonic region, Score2 is assigned a value of 0; if the variant is located in a region selected from a group consisting of UTR-3 region, UTR-5 region, upstream region, downstream region, intronic region, intergenic region, and ncRNA region, Score2 is assigned a value of −1; if the variant is located in a splicing region, Score2 is assigned a value of −2; and
Score3 is evaluated based on the following standard: if the variant is a high-confidence predicted loss-of-function variant, Score3 is assigned a value of +3; if the variant is a low-confidence pLoF variant, Score3 is assigned a value of +2.

5. The method of reducing an artefact variant in high-throughput sequencing of claim 3, wherein in the step of scoring a variant site, a value of M is 100.

6. The method of reducing an artefact variant in a high-throughput sequencing of claim 3, wherein in the step of scoring a variant site, if the score of the variant site is greater than 0, the score of the variant site is assigned a value of 0.

7. The method of reducing an artefact variant in high-throughput sequencing of claim 1, wherein a position of refGene is determined by aligning the position of ref0ene with a master transcript region of NCBI refGene, and for pLoF variants, only Stop-gained variant sites, Splicing variant sites, and Frameshift variant sites found in the master transcript are retained.

8. The method of reducing an artefact variant in high-throughput sequencing of claim 7, wherein the master transcript is a most recently updated transcript in the NCBI refGene.

9. The method of reducing an artefact variant in high-throughput sequencing process of claim 1, wherein in the step of scoring the region sequence, the window size is determined as follows: the window size corresponds to a length of a fragment interval that covers 95±5% of adjacent variant sites in the population variant frequency database.

10. The method of reducing an artefact variant in high-throughput sequencing of claim 9, wherein the database is at least one of dbSNP database, ECAC03 whole exon database and genomeAD whole exon database.

11. The method of reducing an artefact variant in high-throughput sequencing of claim 9, wherein the window size is set to 25±5 bp.

12. The method of reducing an artefact variant in high-throughput sequencing of claim 1, wherein in the step of excluding a variant site, the predetermined threshold value is determined as a top 5% total score of the region sequence when sorting the total score of each region sequence in a sample to be tested from low to high.

13. (canceled)

14. The method of reducing an artefact variant in high-throughput sequencing of claim 1, wherein the high-throughput sequencing is whole exome sequencing.

15. A method of diagnosing and treating a disease, and screening a variant within whole exome, comprising a step of applying the method of reducing an artefact variant in high-throughput sequencing of claim 1.

16. A device for reducing an artefact variant in high-throughput sequencing, comprising:
a module of analysis, configured for analyzing variant data obtained from the high-throughput sequencing data of a sample to be tested using the method of reducing an artefact variant in high-throughput sequencing of claim 1.


SEQUENCE LISTING (100 sequences)

SEQ ID NO: 1 - DNA, 12 bp, Artificial Sequence
  Variant base
ccaccaccacca

SEQ ID NO: 2 - DNA, 102 bp, Artificial Sequence
  Variant base
ccaccaccaccaccacccccaccaccacccccaccacacccctcaccacctccaccaccaccaccacccccaccaccacccccgccacacccctcaccacct

SEQ ID NO: 3 - DNA, 51 bp, Artificial Sequence
  Variant base
ccaccaccaccaccacccccaccaccacccccgccacacccctcaccacct

SEQ ID NO: 4 - DNA, 45 bp, Artificial Sequence
  Variant base
ccaccaccaccaccacccccacccccgccacacccctcaccacct

SEQ ID NO: 5 - DNA, 42 bp, Artificial Sequence
  Variant base
ccaccaccaccacccccacccccgccacacccctcaccacct

SEQ ID NO: 6 - DNA, 84 bp, Artificial Sequence
  Variant base
ccaccaccaccacccccacccccgccacacccctcaccacctccaccaccaccacccccacccccgccacacccctcaccacct

SEQ ID NO: 7 - DNA, 48 bp, Artificial Sequence
  Variant base
ccaccacctccaccaccaccaccaccacccccgccacacccctcacca

SEQ ID NO: 8 - DNA, 57 bp, Artificial Sequence
  Variant base
ccaccacctccaccaccaccaccaccacccccgccacacccctcaccacctccacca

SEQ ID NO: 9 - DNA, 12 bp, Artificial Sequence
  Variant base
ccacctccacca

SEQ ID NO: 10 - DNA, 18 bp, Artificial Sequence
  Variant base
ccacctccaccaccacca

SEQ ID NO: 11 - DNA, 12 bp, Artificial Sequence
  Variant base
ccactaccacca

SEQ ID NO: 12 - DNA, 71 bp, Artificial Sequence
  Variant base
ccccgccacacccctcaccacctccaccaccaccacccccgccacacccctcaccacctccaccaccacca

SEQ ID NO: 13 - DNA, 12 bp, Artificial Sequence
  Variant base
ctaccaccacca

SEQ ID NO: 14 - DNA, 11 bp, Artificial Sequence
  Reference base
caccaccacca

SEQ ID NO: 15 - DNA, 39 bp, Artificial Sequence
  Variant base
ccaccaccaccaccaccaccaccacacccctcaccacct

SEQ ID NO: 16 - DNA, 51 bp, Artificial Sequence
  Variant base
ccaccaccaccaccaccaccaccaccatccccgccacacccctcaccacct

SEQ ID NO: 17 - DNA, 48 bp, Artificial Sequence
  Variant base
ccaccaccaccaccaccaccaccacccccaccacacccctcaccacct

SEQ ID NO: 18 - DNA, 36 bp, Artificial Sequence
  Variant base
ccaccaccaccaccaccaccaccacccccaccacct

SEQ ID NO: 19 - DNA, 69 bp, Artificial Sequence
  Variant base
ccaccaccaccaccaccaccaccacccccacccccaccgccaccatccccgccacacccctcaccacct

SEQ ID NO: 20 - DNA, 54 bp, Artificial Sequence
  Variant base
ccaccaccaccaccaccaccaccacccccacccccgccacacccctcaccacct

SEQ ID NO: 21 - DNA, 63 bp, Artificial Sequence
  Variant base
ccaccaccaccaccaccaccaccacccccaccgccaccatccccgccacacccctcaccacct

SEQ ID NO: 22 - DNA, 105 bp, Artificial Sequence
  Variant base
ccaccaccaccaccaccaccaccacccccaccgccaccatccccgccacacccctcaccacctccaccaccaccaccaccacccccgccacacccctcaccacct

SEQ ID NO: 23 - DNA, 48 bp, Artificial Sequence
  Variant base
ccaccaccaccaccaccaccaccacccccgccacacccctcaccacct

SEQ ID NO: 24 - DNA, 48 bp, Artificial Sequence
  Variant base
ccaccaccaccaccaccaccaccatccccgccacgcccctcaccacct

SEQ ID NO: 25 - DNA, 87 bp, Artificial Sequence
  Variant base
ccaccaccaccaccaccaccacccccgccacacccctcaccacctccaccaccaccaccaccacccccgccacacccctcaccacct

SEQ ID NO: 26 - DNA, 63 bp, Artificial Sequence
  Variant base
ccaccaccaccaccaccacccccacccccaccgccaccacccccgccacacccctcaccacct

SEQ ID NO: 27 - DNA, 63 bp, Artificial Sequence
  Variant base
ccaccaccaccaccaccacccccacccccaccgccaccatccccgccacacccctcaccacct

SEQ ID NO: 28 - DNA, 45 bp, Artificial Sequence
  Variant base
ccaccaccaccaccaccacccccaccgccacacccctcaccacct

SEQ ID NO: 29 - DNA, 57 bp, Artificial Sequence
  Variant base
ccaccaccaccaccaccacccccaccgccaccatccccgccacacccctcaccacct

SEQ ID NO: 30 - DNA, 42 bp, Artificial Sequence
  Variant base
ccaccaccaccaccaccacccccgccacacccctcaccacct

SEQ ID NO: 31 - DNA, 39 bp, Artificial Sequence
  Variant base
ccaccaccaccaccaccaccgccacacccctcaccacct

SEQ ID NO: 32 - DNA, 51 bp, Artificial Sequence
  Variant base
ccaccaccaccaccaccaccgccaccatccccgccacacccctcaccacct

SEQ ID NO: 33 - DNA, 39 bp, Artificial Sequence
  Variant base
ccaccaccaccaccacccccaccacacccctcaccacct

SEQ ID NO: 34 - DNA, 90 bp, Artificial Sequence
  Variant base
ccaccaccaccaccacccccaccatccccgccacacccctcaccacctccaccaccaccaccaccacccccgccacacccctcaccacct

SEQ ID NO: 35 - DNA, 60 bp, Artificial Sequence
  Variant base
ccaccaccaccaccacccccacccccaccgccaccatccccgccacacccctcaccacct

SEQ ID NO: 36 - DNA, 39 bp, Artificial Sequence
  Variant base
ccaccaccaccaccacccccgccacacccctcaccacct

SEQ ID NO: 37 - DNA, 81 bp, Artificial Sequence
  Variant base
ccaccaccaccaccacccccgccacacccctcaccacctccaccaccaccaccaccacccccgccacacccctcaccacct

SEQ ID NO: 38 - DNA, 78 bp, Artificial Sequence
  Variant base
ccaccaccaccaccacccccgccacacccctcaccacctccaccaccaccaccacccccgccacacccctcaccacct

SEQ ID NO: 39 - DNA, 54 bp, Artificial Sequence
  Variant base
ccaccaccaccaccacccccgccacacccctcaccacctccaccaccaccaccc

SEQ ID NO: 40 - DNA, 81 bp, Artificial Sequence
  Variant base
ccaccaccaccaccacccccgccacacccctcaccacctccaccaccaccacccccacccccgccacacccctcaccacct

SEQ ID NO: 41 - DNA, 90 bp, Artificial Sequence
  Variant base
ccaccaccaccaccacccccgccacacccctcaccacctccaccaccaccacccccaccgccaccatccccgccacacccctcaccacct

SEQ ID NO: 42 - DNA, 75 bp, Artificial Sequence
  Variant base
ccaccaccaccaccacccccgccacacccctcaccacctccaccaccaccacccccgccacacccctcaccacct

SEQ ID NO: 43 - DNA, 39 bp, Artificial Sequence
  Variant base
ccaccaccaccaccacccccgccacacccctcaccccct

SEQ ID NO: 44 - DNA, 51 bp, Artificial Sequence
  Variant base
ccaccaccaccaccacccccgccaccatccccgccacacccctcaccacct

SEQ ID NO: 45 - DNA, 93 bp, Artificial Sequence
  Variant base
ccaccaccaccaccacccccgccaccatccccgccacacccctcaccacctccaccaccaccaccaccacccccgccacacccctcaccacct

SEQ ID NO: 46 - DNA, 48 bp, Artificial Sequence
  Variant base
ccaccaccaccaccaccgccaccatccccgccacacccctcaccacct

SEQ ID NO: 47 - DNA, 15 bp, Artificial Sequence
  Variant base
ccaccaccaccaccc

SEQ ID NO: 48 - DNA, 36 bp, Artificial Sequence
  Variant base
ccaccaccaccacccccaccacacccctcaccacct

SEQ ID NO: 49 - DNA, 66 bp, Artificial Sequence
  Variant base
ccaccaccaccacccccaccaccaccacccccaccgccaccatccccgccacacccctcaccacct

SEQ ID NO: 50 - DNA, 51 bp, Artificial Sequence
  Variant base
ccaccaccaccacccccaccaccaccacccccgccacacccctcaccacct

SEQ ID NO: 51 - DNA, 48 bp, Artificial Sequence
  Variant base
ccaccaccaccacccccaccaccacccccaccacacccctcaccacct

SEQ ID NO: 52 - DNA, 57 bp, Artificial Sequence
  Variant base
ccaccaccaccacccccaccaccacccccaccacccccgccacacccctcaccacct

SEQ ID NO: 53 - DNA, 48 bp, Artificial Sequence
  Variant base
ccaccaccaccacccccaccaccacccccgccacacccctcaccacct

SEQ ID NO: 54 - DNA, 39 bp, Artificial Sequence
  Variant base
ccaccaccaccaccccccccgccacacccctcaccacct

SEQ ID NO: 55 - DNA, 36 bp, Artificial Sequence
  Variant base
ccaccaccaccacccccgccacacccctcaccacct

SEQ ID NO: 56 - DNA, 78 bp, Artificial Sequence
  Variant base
ccaccaccaccacccccgccacacccctcaccacctccaccaccaccaccaccacccccgccacacccctcaccacct

SEQ ID NO: 57 - DNA, 120 bp, Artificial Sequence
  Variant base
ccaccaccaccacccccgccaccccccccaccacctccacccccaccaccaccacccccgccacacccctcaccacctccaccaccaccaccaccacccccgccacacccctcaccacct

SEQ ID NO: 58 - DNA, 69 bp, Artificial Sequence
  Variant base
ccaccaccaccacctccaccaccaccaccacccccaccgccaccatccccgccacacccctcaccacct

SEQ ID NO: 59 - DNA, 12 bp, Artificial Sequence
  Variant base
ccaccaccaccc

SEQ ID NO: 60 - DNA, 45 bp, Artificial Sequence
  Variant base
ccaccaccacccccaccaccacccccaccacacccctcaccacct

SEQ ID NO: 61 - DNA, 39 bp, Artificial Sequence
  Variant base
ccaccaccacccccacccccgccacacccctcaccacct

SEQ ID NO: 62 - DNA, 81 bp, Artificial Sequence
  Variant base
ccaccaccacccccacccccgccacacccctcaccacctccaccaccaccaccaccaccaccgccacacccctcaccacct

SEQ ID NO: 63 - DNA, 81 bp, Artificial Sequence
  Variant base
ccaccaccacccccacccccgccacacccctcaccacctccaccaccaccaccaccacccccgccacacccctcaccacct

SEQ ID NO: 64 - DNA, 18 bp, Artificial Sequence
  Variant base
ccaccaccacccccaccg

SEQ ID NO: 65 - DNA, 78 bp, Artificial Sequence
  Variant base
ccaccaccacccccaccgccacacccctcaccacctccaccaccaccaccaccacccccgccacacccctcaccacct

SEQ ID NO: 66 - DNA, 48 bp, Artificial Sequence
  Variant base
ccaccaccacccccaccgccaccacccccgccacacccctcaccacct

SEQ ID NO: 67 - DNA, 48 bp, Artificial Sequence
  Variant base
ccaccaccacccccaccgccaccatccccgccacacccctcaccacct

SEQ ID NO: 68 - DNA, 90 bp, Artificial Sequence
  Variant base
ccaccaccacccccaccgccaccatccccgccacacccctcaccacctccaccaccaccaccaccacccccgccacacccctcaccacct

SEQ ID NO: 69 - DNA, 87 bp, Artificial Sequence
  Variant base
ccaccaccacccccaccgccaccatccccgccacacccctcaccacctccaccaccgccaccatccccgccacacccctcaccacct

SEQ ID NO: 70 - DNA, 48 bp, Artificial Sequence
  Variant base
ccaccaccacccccaccgccaccatccccgccacgcccctcaccacct

SEQ ID NO: 71 - DNA, 33 bp, Artificial Sequence
  Variant base
ccaccaccacccccgccacacccctcaccacct

SEQ ID NO: 72 - DNA, 12 bp, Artificial Sequence
  Variant base
ccaccaccacct

SEQ ID NO: 73 - DNA, 51 bp, Artificial Sequence
  Variant base
ccaccacccccacccccaccgccaccatccccgccacacccctcaccacct

SEQ ID NO: 74 - DNA, 72 bp, Artificial Sequence
  Variant base
ccaccacccccgccacacccctcaccacctccaccaccaccaccaccacccccgccacacccctcaccacct

SEQ ID NO: 75 - DNA, 39 bp, Artificial Sequence
  Variant base
ccaccaccgccaccatccccgccacacccctcaccacct

SEQ ID NO: 76 - DNA, 18 bp, Artificial Sequence
  Variant base
ccaccacctccaccacct

SEQ ID NO: 77 - DNA, 39 bp, Artificial Sequence
  Variant base
ccacccccaccaccacccccgccacacccctcaccacct

SEQ ID NO: 78 - DNA, 48 bp, Artificial Sequence
  Variant base
ccacctccaccaccaccaccaccacccccgccacacccctcaccacct

SEQ ID NO: 79 - DNA, 99 bp, Artificial Sequence
  Variant base
ccacctccacccccaccaccaccacccccgccacacccctcaccacctccaccacctccaccaccaccaccaccacccccgccacacccctcaccacct

SEQ ID NO: 80 - DNA, 72 bp, Artificial Sequence
  Variant base
caccaccaccaccaccaccaccacccccaccacacccctcaccacctccaccaccacctccaccacctccac

SEQ ID NO: 81 - DNA, 30 bp, Artificial Sequence
  Variant base
caccaccaccaccacccccgccacacccct

SEQ ID NO: 82 - DNA, 18 bp, Artificial Sequence
  Variant base
caccaccaccacctccac

SEQ ID NO: 83 - DNA, 12 bp, Artificial Sequence
  Variant base
caccacctccac

SEQ ID NO: 84 - DNA, 21 bp, Artificial Sequence
  Variant base
caccacctccaccacctccac

SEQ ID NO: 85 - DNA, 63 bp, Artificial Sequence
  Variant base
caccacctccacccccaccaccacccccaccacacccctcaccacctccaccaccacctccac

SEQ ID NO: 86 - DNA, 21 bp, Artificial Sequence
  Reference base
ccaccaccaccacccccaccg

SEQ ID NO: 87 - DNA, 12 bp, Artificial Sequence
  Variant base
accaccaccacc

SEQ ID NO: 88 - DNA, 54 bp, Artificial Sequence
  Variant base
ccaccaccaccacccccgccacacccctcaccacctccaccaccaccaccaccc

SEQ ID NO: 89 - DNA, 39 bp, Artificial Sequence
  Variant base
ccaccaccaccacccccgccacacccctcaccacctccc

SEQ ID NO: 90 - DNA, 12 bp, Artificial Sequence
  Variant base
aaaaaaaaacaa

SEQ ID NO: 91 - DNA, 13 bp, Artificial Sequence
  Reference base
aaaaaaaaaacaa

SEQ ID NO: 92 - DNA, 23 bp, Artificial Sequence
  Reference base
aaaaaaaaaacaaaaaaaaaaac

SEQ ID NO: 93 - DNA, 22 bp, Artificial Sequence
  Reference bas
aaaaaaaaacaaaaaaaaaaac

SEQ ID NO: 94 - DNA, 21 bp, Artificial Sequence
  Reference bas
aaaaaaaacaaaaaaaaaaac

SEQ ID NO: 95 - DNA, 20 bp, Artificial Sequence
  Reference bas
aaaaaaacaaaaaaaaaaac

SEQ ID NO: 96 - DNA, 19 bp, Artificial Sequence
  Reference bas
aaaaaacaaaaaaaaaaac

SEQ ID NO: 97 - DNA, 18 bp, Artificial Sequence
  Reference bas
aaaaacaaaaaaaaaaac

SEQ ID NO: 98 - DNA, 16 bp, Artificial Sequence
  Reference bas
aaacaaaaaaaaaaac

SEQ ID NO: 99 - DNA, 15 bp, Artificial Sequence
  Reference bas
aaaaaaaaaaacaaa

SEQ ID NO: 100 - DNA, 13 bp, Artificial Sequence
  Reference bas
aaaaaaaaacaaa